(19) United States
(12) Patent Application Publication
(10) Pub. No.: US 2023/0352123 A1
(43) Pub. Date: Nov. 2, 2023

(54) AUTOMATIC DESIGN OF MOLECULES HAVING SPECIFIC DESIRABLE CHARACTERISTICS

(71) Applicant: NNAISENSE, SA, Lugano (CH)

(72) Inventors: Timothy Atkinson, York (GB); Saeed Saremi, San Francisco, CA (US); Jonatan Masci, Lugano (CH)

(21) Appl. No.: 18/041,807

(22) PCT Filed: Aug. 17, 2021
(86) PCT No.: PCT/US2021/046391

§ 371 Date: Feb. 16, 2023

Related U.S. Application Data
(60) Provisional application No. 63/067,121, filed on Aug. 18, 2020.

(51) Int. Cl. G16C 20/30; G16C 20/50; G16C 20/70; G06N 3/045

(52) U.S. Cl. CPC G16C 20/30; G16C 20/50; G16C 20/70; G06N 3/045

(57) ABSTRACT

A computer system is disclosed for designing molecules with a desired property (e.g., the ability to inhibit SARS-CoV-2). The computer system includes a computer-based processor and a computer-readable medium storing computer-readable instructions that, when executed by the computer-based processor, cause the computer-based processor to embody: a computer-based search engine configured to search for proposed molecules, a computer-based property predictor to predict a likelihood of the desired property being present in the proposed molecules, and a computer-based energy model to approximate a degree of similarity between the proposed molecules and molecules known to possess the desired property. The search is guided by the property predictor's predictions and the energy model's approximations.

### CROSS-REFERENCE TO RELATED APPLICATIONS

The present application claims the benefit of priority to U.S. Provisional Patent Application No. 63/067,121, entitled Automatic Design of Novel Potential 3CL^{pro} and PL^{pro} Inhibitors, which was filed on Aug. 18, 2020. The disclosure in the prior application is incorporated by reference herein in its entirety.

### FIELD OF THE INVENTION

This disclosure relates to the automatic design of molecules having specific desirable characteristics, such as novel potential 3CL^{pro} AND PL^{pro} inhibitors, and, more particularly, relates to computer-based systems and techniques for automatically designing same.

### BACKGROUND

In recent history, the search for molecules which may inhibit key receptor sites of Severe Acute Respiratory Syndrome Coronavirus-2 (SARS-CoV-2), for example, has emerged as a central research objective within the scientific community.

A need exists for enhanced systems and methods to identify or design such molecules as well as others.

### SUMMARY OF THE INVENTION

In one aspect, a computer system is disclosed for designing molecules with a desired property (e.g., the ability to inhibit SARS-CoV-2). The computer system includes a computer-based processor and a computer-readable medium storing computer-readable instructions that, when executed by the computer-based processor, cause the computer-based processor to embody: a computer-based search engine configured to search for proposed molecules, a computer-based property predictor to predict a likelihood of the desired property being present in the proposed molecules, and a computer-based energy model to approximate a degree of similarity between the proposed molecules and molecules known to possess the desired property.

The search is guided by the property predictor's predictions and the energy model's approximations.

In another aspect, a computer-based method is disclosed for designing molecules having a desired property (e.g., ability to inhibit SARS-CoV-2). The computer-based method includes: providing a computer system that has a computer-based processor, and a computer-readable medium storing computer-readable instructions that, when executed by the computer-based processor, cause the computer-based processor to embody: a computer-based property predictor to predict a likelihood that each respective one of a plurality of proposed molecules has the desired property, a computer-based energy model to approximate a statistical similarity between each respective one of the proposed molecules and a set of molecules represented in a stored dataset of training molecules (the dataset of training molecules contains data regarding a set of molecules known to possess the desired property), and a computer-based search engine to conduct a search through a space of candidate molecules to propose molecules likely to have the desired property, wherein the search is guided by the property predictor's predictions and by the energy model's approximations.

In yet another aspect, a non-transitory computer readable medium having stored thereon computer-readable instructions that, when executed by a computer-based processor, cause the computer-based processor to embody: a computer-based property predictor that predicts a likelihood that each respective one of a plurality of proposed molecules has the desired property, a computer-based energy model that approximates a statistical similarity between each respective one of the proposed molecules and a set of molecules represented in a stored dataset of training molecules, wherein the dataset of training molecules contains data regarding a set of molecules known to possess the desired property, and a computer-based search engine that conducts a search through a space of candidate molecules to propose molecules likely to have the desired property, wherein the search is guided by the property predictor's predictions and by the energy model's approximations.

In some implementations, one or more of the following advantages are present.

For example, in various implementations, the systems and techniques disclosed herein facilitate and provide for automatic design of novel potential 3CL^{pro} AND PL^{pro} inhibitors. Moreover, in a typical implementation, this process can yield results quickly and efficiently.

Additionally, there exists a dataset/multiple datasets of molecules labeled with numerical or categorical information describing desirable properties such as; efficacy or activity with respect to a medical function, toxicity, drug likeness and synthetic accessibility. It may be possible to build machine learning models to model these desirable properties, but due to the large combinatorial space of small molecules, this cannot provide trustworthy predictions on out-of-distribution (with respect to the training dataset(s)) molecules. Out-of-distribution is a somewhat hard-to-define term, but here it means that molecules that are very close in structure to the molecules in the training set can be considered in-distribution. Molecules with more and more differences in structure gradually become more and more out-of-distribution.

Some search methods can optimize for desirable properties using a trained machine learning model as a heuristic/approximation for molecules' true numerical or categorical values of those properties. Search methods applied in this way cannot generally function effectively once they leave the distribution of the training dataset(s) molecules, when the predictions made by the machine learning approximation are no longer trustworthy. Moreover, the distribution of the training dataset(s) molecules may not be formally known, and the distribution of the training dataset(s) generally cannot be identified by simple analytic methods.

In some implementations, integration of an energy model into the system and search procedures disclosed herein allows the search procedure to target areas of the vast space of which statistically resemble the original training data, increasing the confidence of the property predictor(s). Other ways of resolving the issue of confidence with property prediction may include: 1) performing a search against simulations, rather than trained models (simulations are reliable and naturally generalize well but require experts for their construction), and 2) training a model, and then using it to filter a larger dataset of known similar molecules (this prevents more generic search and requires that a larger dataset of known similar molecules is already available). Various implementations of the systems and techniques disclosed herein address these (and other) shortcomings.

Other approaches explore search against a trained ML model e.g. as a discriminator to improve a genetic algorithm's mutations or as a predictive model of properties. These other approaches, however, do not incorporate a mechanism similar to the energy model (MONAS-E) disclosed herein for discriminating between statistically similar and distinct molecules. In fact, in some implementations, the energy model (MONAS-E) may facilitate finding statistical overlaps between multiple datasets.

Other features and advantages will be apparent from the description and drawings, and from the claims.

### BRIEF DESCRIPTION OF THE DRAWINGS

FIG. 1 is a schematic representation of an exemplary computer-based system that provides for the design of novel potential 3CL^{pro} AND PL^{pro} inhibitors.

FIG. 2 is a schematic representation of an exemplary computer network environment that may be used to implement functionalities disclosed herein including facilitating and providing for automatic design of novel potential 3CL^{pro} AND PL^{pro} inhibitors.

FIG. 3 is a schematic representation showing a detailed implementation of the network environment represented in FIG. 2.

FIG. 4 is a schematic representation showing components of an exemplary design platform from the network in FIGS. 2 and 3 as well as dataflow between those components according to one exemplary embodiment.

FIG. 5 is a flowchart with an exemplary set of computer system functionalities involved in automatically designing molecules having particular characteristics (e.g., novel potential 3CL^{pro} AND PL^{pro} inhibitors).

FIG. 6 is a flowchart with an exemplary set of computer system functionalities involved in formatting molecular data from a training set into a graph format.

FIG. 7 is a schematic representation showing an exemplary architecture of a property predictor.

FIG. 8 is a schematic representation of a Deep Energy Estimator Network (DEEN) process.

FIGS. 9A and 9B are schematic representations showing Monte Carlo tree searching (“MCTS”) steps.

FIGS. 10A and 10B, respectfully, show validation and testing receiver operator characteristic (ROC) area under curve (AUC) scores of a model measured during training and reported for assays.

FIG. 11 shows DEEN model learning curves that indicate the training and testing loss measured during multiple training epochs.

FIGS. 12A, 12B, and 12C are molecular diagrams showing eight known inhibitors (12A) in comparison to eight inhibitors discovered with (12B) and without (12C) energy regularization.

FIG. 13 is a chart that compares the energies (as computed by an energy model) of various discovered inhibitors with and without energy regularization and known (testing) inhibitors.

FIG. 14 is a chart of energy vs. synthetic accessibility for an assay.

Like reference characters refer to like elements.

### DETAILED DESCRIPTION

This document uses a variety of terminology to describe its inventive concepts. Unless otherwise indicated, the following terminology, and variations thereof, should be understood as having meanings that are consistent with what follows.

For example, unless otherwise indicated, the phrase “coronavirus” refers to any of a group of ribonucleic acid (RNA) viruses that cause a variety of respiratory, gastrointestinal, and neurological diseases in humans and other animals.

Unless otherwise indicated, the phrases “SARS-CoV-1” and “SARS-CoV-2” refer, respectively, to severe acute respiratory syndrome coronaviruses, which are strains of coronavirus that cause severe acute respiratory syndrome (SARS).

Unless otherwise indicated, the phrase “protease” refers to any of numerous enzymes that catalyze, for example, the hydrolytic breakdown proteins.

Unless otherwise indicated, the phrases “3CL^{pro}” and “PL^{pro}” refer to proteases found in coronavirus and are critical for SARS-CoV-2 replication.

Unless otherwise indicated, the phrase “inhibitor” refers to a substance that reduces or suppresses the activity of another substance (e.g., an enzyme, such as a protease).

Unless otherwise indicated, the phrase “predictive model” refers to a computer-based component that predict outcomes (e.g., likelihood that a particular molecule will be an effective inhibitor), typically, based on statistics.

Unless otherwise indicated, the phrase “artificial neural network” (also simply “neural network”) refers to a computing system that includes a plurality of connected units (“nodes”) that act as artificial neurons. Each connection can be used to transmit a signal from one node to another node. A node that receives a signal then processes it and can signal other nodes connected to it. The signal at a connection generally represents a real number value, and the output of each node is generally computed based on a function (e.g., a non-linear function of the sum of its inputs). The connections between nodes are called edges. Each node and each edge can have an associated weight (e.g., a numerical value) that adjusts as learning/training proceeds. The weight increases or decreases the strength of the signal at a connection. Typically, nodes are aggregated into layers and different layers may perform different transformations on their inputs. Signals generally travel from a first layer (i.e., an input layer), to the last layer (i.e., an output layer), possibly after traversing the layers multiple times.

Unless otherwise indicated, the phrase “graph neural network” refers to a class of neural networks for processing data represented by graph data structures.

Unless otherwise indicated, the phrase “graph data structure” refers to a computer-based data structure that consists of a finite (and possibly mutable) set of vertices (also called nodes or points), together with a set of unordered pairs of these vertices for an undirected graph or a set of ordered pairs for a directed graph. These pairs are known as edges (also called links or lines), and for a directed graph are also known as edges but also sometimes arrows or arcs. The vertices may be part of the graph structure or may be external entities represented by integer indices or references. A graph data structure may also associate to each edge some edge value, such as a symbolic label or a numeric attribute.

Unless otherwise indicated, the phrase “supervised learning” refers to a process by which neural networks can learn (or be trained) by processing examples, each of which contains a known “input” and “result,” and forming probability-weighted associations between the two, which are stored within the data structure of the network itself. The training of a neural network from a given example is usually conducted by determining the difference between the processed output of the network (often a prediction) and a target output. This difference is an error. The network then adjusts its weighted associations according to a learning rule and using this error value. Successive adjustments will cause the neural network to produce output which is increasingly similar to the target output. After a sufficient number of these adjustments the training can be terminated based upon certain criteria.

Unless otherwise indicated, the phrase “unsupervised learning” refers to a process by which a neural network can learn (or be trained to identify) patterns from untagged data. In unsupervised learning, the hope is that the machine is forced to learn a compact internal representation of the patterns of its world which may then be used for various tasks such as generating imaginative content and automatically grouping individual data points. In contrast to supervised learning where data is tagged by a human (e.g., as “car” or “fish” etc.), unsupervised learning exhibits self-organization that captures patterns as nodal predilections or probability densities.

Unless otherwise indicated, the phrase “probabilistic model” refers to a computer-based model that incorporates random variables and/or probability distributions into a model of an event or phenomenon. While a deterministic model generally produces a single possible outcome for an event, a probabilistic model generally produces a probability distribution as a solution.

Unless otherwise indicated, the term “scalar” represents a real number, rather than a vector, for example.

Unless otherwise indicated, the phrase “statistical similarity” refers to a statistical measure of similarity (e.g., between a proposed new molecule and molecule(s) known to be inhibitors). In statistics, for example, a similarity measure or similarity function is a real-valued function that quantifies the similarity between two objects (e.g., between a molecule and one or more other molecules). Such measures may be thought of as the inverse of a distance metric (e.g., they take on large values for highly similar objects and either zero or a negative value for very dissimilar objects.)

Unless otherwise indicated, the phrase “directed search” (or simply “search”) refers to a computer-based search. Typically:
there is at least one function to optimize (maximize/minimize),
the search method proposes solutions which are evaluated against the function(s) to optimize,
the results of the evaluations are used by the search method in the next iteration of generating solutions.

Unless otherwise indicated, the phrase “Deep Energy Estimator Network” or “DEEN” refers to a computer-based energy function network, an example of which was described in a publication by S. Saremi, A. Mehrjou, B. Scholkopf, and A. Hyvarinen, entitled Deep energy estimator networks. (arXiv preprint arXiv:1805.08306, 2018). In a typical implementation, a DEEN is well-suited for density estimation particularly in applications involving for complex high-dimensional data.

Unless otherwise indicated, the phrase “Monte Carlo tree searching” refers to a heuristic search algorithm for decision processes. In a typical implementation, Monte Carlo tree searching may involve the iterative rounds of steps including selection, expansion, simulation, and backpropagation.

Unless otherwise indicated, the phrase “Backus-Naur form” and the like refers to a metasyntax notation for context-free grammars. More specifically, it refers to a syntactic metalanguage (i.e., a language about a language). The metalanguage is a formal notation for specifying the grammar that describes the syntax of a programming language.

Unless otherwise indicated, the phrase “Long Short-Term Memory” or “LSTM” refers to a computer-based artificial recurrent neural network (“RNN”) architecture used in the field of deep learning.

Unless otherwise indicated, the phrase “simplified molecular-input line-entry system” or “SMILES” refers to a form of line notation for describing the structure of chemical species using short American Standard Code for Information Interchange (“ASCII”) strings, or the specification for same. (See, e.g., David Weininger. Smiles, a chemical language and information system. 1. introduction to methodology and encoding rules. Journal of chemical information and computer sciences, 28(1):31-36, 1988.) SMILES strings can be imported by most molecule editors for conversion back into two-dimensional drawings or three-dimensional models of the molecules.

Unless otherwise indicated, the phrase “variational autoencoder” refers to a computer-based component that provides a probabilistic way to describe an observation (e.g., in latent space). In a typical implementation, a variational autoencoder can produce a probability distribution for each attribute.

Unless otherwise indicated, the phrase “genetic algorithm” refers to a computer-based metaheuristic inspired by the process of natural selection that can be used to generate high-quality solutions to optimization and search problems by relying on biologically-inspired operators such as mutation, crossover, and selection for example.

Unless otherwise indicated, the phrase “deep reinforcement learning” refers to a form of machine learning that combines reinforcement learning and deep learning. Reinforcement learning generally considers the problem of a computational agent learning to make decisions by trial and error. Deep reinforcement learning incorporates deep learning into the solution, allowing agents to make decisions, for example, from unstructured input data without manual engineering of the state space. Typically, deep reinforcement learning algorithms are able to take in very large inputs and decide what actions to perform to optimize an objective.

In machine learning and pattern recognition, a feature vector is an n-dimensional vector of numerical features, for example, that represent some object. Many algorithms in machine learning require that objects be represented numerically since such representations can facilitate processing and statistical analysis. Unless otherwise indicated, the vector space associated with these types of vectors is referred to as a “feature space.” Unless otherwise indicated, the phrase “combinatorial space” refers to a space constructed as a combination of simpler spaces. Suppose, for example, the space [A, B, C] and the space [1, 2, 3]. A combinatorial space constructed as a cartesian product of these spaces is [[A,1], [A,2], [A,3], [B,1], [B,2], [B,3], [C, 1], [C,2], [C,3]]. Small molecules can be viewed as a combinatorial space of combinations of individual atoms and bonds. Note that, unlike the above simple cartesian space, the molecular search space is not all combinations of individual atoms and bonds as some (many) are impossible by the laws of chemistry.

Unless otherwise indicated, the term “PubChem” refers to a key chemical information resource for the biomedical research community.

Unless otherwise indicated, the term “assay” refers to an examination and determination of characteristics (functional and/or structural) of an entity or refers to the tabulated results of such an examination and determination.

Unless otherwise indicated, the phrase, “batch normalization” refers to a computer-based method used to make artificial neural networks faster and more stable through normalization of the layers' inputs, for example, by re-centering and re-scaling.

Unless otherwise indicated, the phrase “rectified linear unit” or “ReLU” activation function or the like refers to an activation function defined as the positive part of its argument, for example:

ƒ(x)=x⁺=max(0,x)

where x is the input to a neuron.

Unless otherwise indicated, the phrase “reward function” or the like refers to a computer-implemented function that provides a score (e.g., numerical in nature) based on the state of an environment. In essence, in a typical implementation, a reward function can be thought of as a mapping of each perceived state of the environment to a single number, specifying the intrinsic desirable of that state.

Unless otherwise indicated, the term “backpropagating” and the like refers to computing, in a neural network, a gradient of a loss function with respect to weights of the network for a single input-output example. Backpropagation algorithms typically work by computing the gradient of the loss function with respect to each weight by the chain rule, computing the gradient one layer at a time, iterating backward from the last layer to avoid redundant calculations of intermediate terms in the chain rule.

### Prior Technologies

Prior approaches exist to identify SARS-CoV-2 inhibitors, for example. Some of the existing approaches, for example, use deep learning to predict inhibition in order to screen sets of known inhibitors.

The systems and techniques disclosed herein, however, differ from prior approaches and solve various technical shortcomings associated with those prior approaches, particularly as it relates to designing SARS-CoV-2 inhibitors, for example. The systems and techniques disclosed herein provide a de novo approach for designing novel inhibitors for SARS-CoV-2 (or other molecules). De novo is the Latin for ‘new’. Specifically, in this context, it refers to the fact that the considered molecules are constructed/generated ‘from scratch’, rather than taking them directly from a dataset. These de novo systems and techniques, in a typical implementation, afford several technical advantages:
1. The combinatorial space of small drug-like molecules is huge (approx. 1033) so that it is unlikely that even datasets with billions of molecules will contain the best SARS-CoV-2 inhibitors. Implementations of the systems and techniques disclosed herein, especially those involving directed searching, provide for searching through the full, or at least a large portion of, combinatorial spaces, which increases the likelihood of yielding high-quality results.
2. Implementations of the systems and techniques disclosed herein may facilitate discovery of potential inhibitors in molecules previously unknown to be inhibitors. If any discovered molecule is eventually tested and found viable for treatment, this may substantially improve the general availability of treatment. Unlike other de novo approaches which utilize docking simulations (See, e.g., Tim Cofala, Lars Elend, Philip Mirbach, Jonas Prellberg, Thomas Teusch, and Oliver Kramer. Evolutionary multi-objective design of sars-cov-2 protease inhibitor candidates. arXiv preprint arXiv:2005.02666, 2020) or structural information (Bowen Tang, Fengming He, Dongpeng Liu, Meijuan Fang, Zhen Wu, and Dong Xu. Ai-aided design of novel targeted covalent inhibitors against sars-cov-2. bioRxiv, 2020), implementations of the systems and techniques disclosed herein increase/maximize the output of a neural model trained to predict SARS-CoV-1 inhibition. The use of a predictive network greatly reduces the cost of sampling so that millions of candidate molecules can be explored efficiently.

As mentioned above, in some implementations, integration of an energy model into the system and search procedures disclosed herein allows the search procedure to target areas of the vast space of which statistically resemble the original training data, increasing the confidence of the property predictor(s). Other ways of resolving the issue of confidence with property prediction may include: 1) performing a search against simulations, rather than trained models (simulations are reliable and naturally generalize well but require experts for their construction), and 2) training a model, and then using it to filter a larger dataset of known similar molecules (this prevents more generic search and requires that a larger dataset of known similar molecules is already available). Various implementations of the systems and techniques disclosed herein address these (and other) shortcomings.

Other approaches explore search against a trained ML model, e.g., as a discriminator to improve a genetic algorithm's mutations or as a predictive model of properties. These other approaches, however, do not incorporate a mechanism similar to the energy model (MONAS-E) disclosed herein for discriminating between statistically similar and distinct molecules. In fact, in some implementations, the energy model (MONAS-E) may facilitate finding statistical overlaps between multiple datasets.

More broadly, implementations of the systems and techniques disclosed herein provide for enhanced efficiently, reliability, and accuracy in designing molecules for particular purposes (including, for example, ability to inhibit SARS-CoV-2, etc.). Implementations of the systems and techniques disclosed herein may enjoy other technical distinctions and advantages over prior technologies.

### Technical Disclosure

With an aim of facilitating the design/identification of novel inhibitors for SARS-CoV-1 and SARS-CoV-2 (and potentially other novel molecules having particular characteristics), the systems and techniques disclosed herein provide a general molecule optimization framework, referred to herein as a Molecular Neural Assay Search (MONAS), that comprises three primary components: a property predictor (also referred to herein as MONAS-P) that identifies molecules with specific desirable properties, an energy model (also referred to herein as MONAS-E) that approximates the statistical similarity of a given molecule to known training molecules, and a molecule search engine (also referred to herein as MONAS-S) that, in some implementations, performs directed searching. In an exemplary implementation, these components are instantiated with graph neural networks (GNNs), Deep Energy Estimator Networks (DEEN) and Monte Carlo tree search (MCTS) techniques, respectively. Such implementations may be used, for example, to identify molecules (e.g., 120K molecules out of 40-million or more explored molecules) that the GNN(s), for example, identify as likely SARS-CoV-1 inhibitors, and also statistically close (or structurally similar) to the training molecules in a dataset used to train the GNN(s).

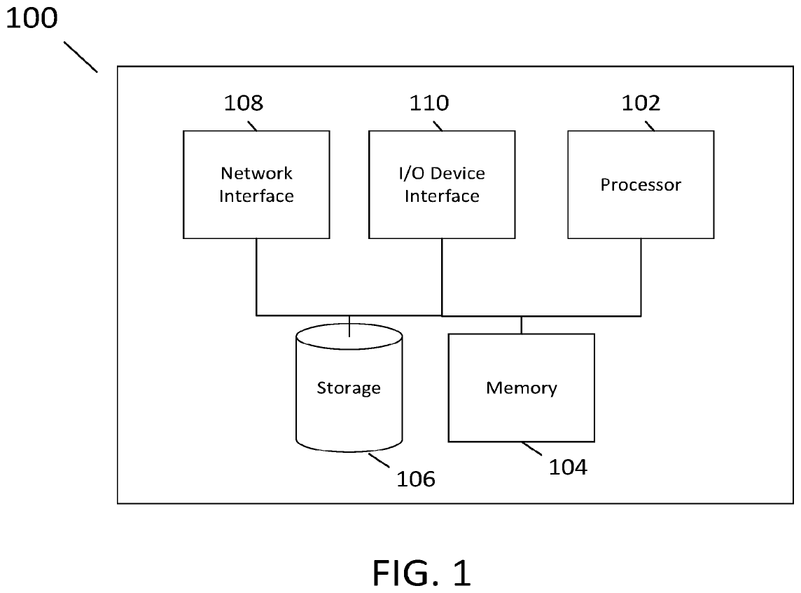

FIG. 1 is a schematic representation of an exemplary computer-based system 100 that provides for the design of novel potential 3CL^{pro} AND PL^{pro} inhibitors. More specifically, in a typical implementation, the computer-system 100 is generally configured to identify from a dataset of proposed novel inhibitors, those molecules that are likely inhibitors and also statistically close to molecules that actually are known inhibitors. In a typical implementation, the computer system 100 performs these, and other related and supporting functionalities, quickly, efficiently and accurately. Moreover, in some implementations, once the datasets (of proposed inhibitor molecules and known inhibitor molecules) are loaded into the system 100 and the design process initiated, the system 100 may operate largely, if not entirely, automatically, to perform the indicated design functionalities.

The computer system 100 has a processor 102 (e.g., CPU), computer-based memory 104, computer-based storage 106, a network interface 108, an input/output device interface 110, and a communications medium/bus that serves as an interconnect between the components of the computer system 100. In a typical implementation, the bus acts as a communications medium over which the various components of the computer system 100 can communicate and interact with one another.

The central processing unit (CPU) 102 is configured to perform various computer-based functionalities disclosed herein related to design of novel inhibitors as well as other supporting functionalities that may or may not be disclosed explicitly herein. Typically, the CPU 102 performs its functionalities by executing computer-readable instructions that may be stored in a non-transient computer-readable medium, such as in the system's computer-based memory 104 and/or computer-based storage 106. In some implementation, the processor's functionalities may be performed by executing computer-readable instructions stored elsewhere (e.g., on an external computer-readable medium) and accessed by the CPU via an I/O device connected to the I/O device interface 110 or the network interface 108.

The illustrated system 100 has both volatile and non-volatile computer-based storage. More specifically, in this regard, the system's computer-based memory 104 provides a form of volatile storage for computer-readable instructions that, when executed by the CPU 102, cause the CPU 102 to perform at least some (or all) of the computer-based functionalities disclosed herein. In some implementations, the system's computer-based memory 104 will also store one or more datasets including for example, a first dataset of information regarding inhibitor molecule and a second dataset of information regarding proposed novel inhibitor molecules. In some implementations, the first dataset may be stored in a first database and the second dataset may be stored in a second database. Computer-based storage 106 provides a form of non-volatile storage for software instructions, such as instructions to implement an operating system, as well as configurations information, etc.

The network interface 108 is a system component that enables connecting to any one or more of a variety of external computer-based communications networks, including, for example, local area networks (LANs), wide area networks (WANs) such as the Internet, or the like. Moreover, the network interface 108 facilitates connecting and communicating with any one of a variety of external computer-based network components, via a communications network.

The input/output device interface 110 is a system component that provides a connection interface for one or more input and/or output devices such as a keyboard, mouse, display, microphone, speakers, printers, etc.

In various implementations, the computer system 100 may have additional elements, such as controllers, buffers (caches), drivers, repeaters, and receivers, to facilitate communications and/or other computer-based functionalities. Furthermore, the interfaces (e.g., 108, 110) may include address, control, and/or data connections to facilitate communication among the illustrated components.

In various implementations, each component of the computer system 100 may be implemented in hardware, in software executing on one or more computer processors, or in a combination of hardware and software.

In a typical implementation, the system 100 is loaded with and configured to run computer-readable instructions (e.g., software) that, when executed by a computer processor (e.g., processor 104) cause the processor to perform or facilitate functionalities disclosed herein related to the design of novel potential 3CL^{pro} AND PL^{pro} inhibitors. In a typical implementation, the software is provided as a set of computer-readable instructions stored in the computer-based memory 104 (or computer-based storage 106), with some of those instructions being stored externally, as an option. The processor 102 executes the computer-readable instructions to perform one or more of the various functionalities associated with the design techniques disclosed herein, alone or in conjunction with other software functionalities.

Moreover, in a typical implementation, one or more computer-based I/O devices (e.g., a computer screen, keyboard, mouse, printer, touch screen device, etc.) may be connected to the I/O device interface 110. The connected I/O devices are generally configured to enable a human user to interact with the system 100 and to access and utilize the functionalities, particularly those related to the design of novel potential inhibitors, disclosed herein.

In an exemplary implementation, the computer system 100 may operate to display (e.g., on a computer-based display device connected to the I/O device interface 110), a visual representation of an interface that provides user access to the functionalities associated with the design of novel potential inhibitors. The interface and its visual representation on the computer-based display device may solicit or enable entry of user-specified parameters. The interface and its visual representation on the computer-based display device may visually display various data associated with designs being created/identified.

The network interface 108 may be connected, via a network, to other system components (e.g., other systems, computer components, servers, etc.) to enable resource and/or data sharing, collaboration, accessing of externally-stored software and/or additional processing functionalities, etc.

In various implementations, the components in system 100 may be contained in one physical device (e.g., a laptop or desktop computer). In various implementations, the components in system 100 may be distributed across more than one physical device (e.g., multiple laptops and/or desktop computers, one or more servers, etc.). For example, the processor 102 may represent a single processor in a single physical device (e.g., a laptop or desktop computer, or a server) or may represent a plurality of processors distributed across multiple physical devices (e.g., one or more laptop computers and/or one or more network servers) working collectively to provide the functionalities disclosed herein. As another example, the memory 104 and/or the storage 106 may be contained within a single physical device (e.g., a laptop or desktop computer or a server), or may be distributed across multiple physical devices connected together via a network. In network configurations where the components of the system are distributed across multiple physical devices (e.g., one or more laptop computers and/or one or more network servers) connected via the network, each discrete physical device may include a network interface 108, one or more I/O device interfaces 110 and/or any one or more of a variety of other computer system components. A wide variety of possibilities regarding specific physical implementations are possible.

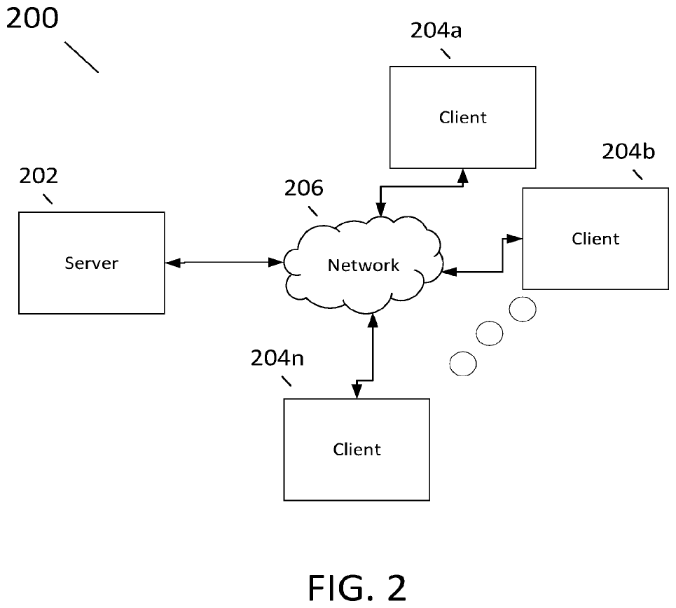

FIG. 2 is a schematic representation of an exemplary computer network environment 200 that may be used to implement the functionalities disclosed herein including, for example, the design functionalities.

The illustrated computer network environment 200 has a server 202, and several client devices 204a, 204b, . . . 204n (or “clients”) coupled to one another via a communications network 206 that enables the server 202 and clients 204a, 204b, . . . 204n to communicate and interact with one another. In various implementations, one or more (or every single one) of the clients 204a, 204b, . . . 204n, may have the same types of components as those shown in the computer system 100 of FIG. 1. Moreover, in various implementations, the server 202 may have one or more (or all) of the same types of components as those shown in the computer system 100 of FIG. 1.

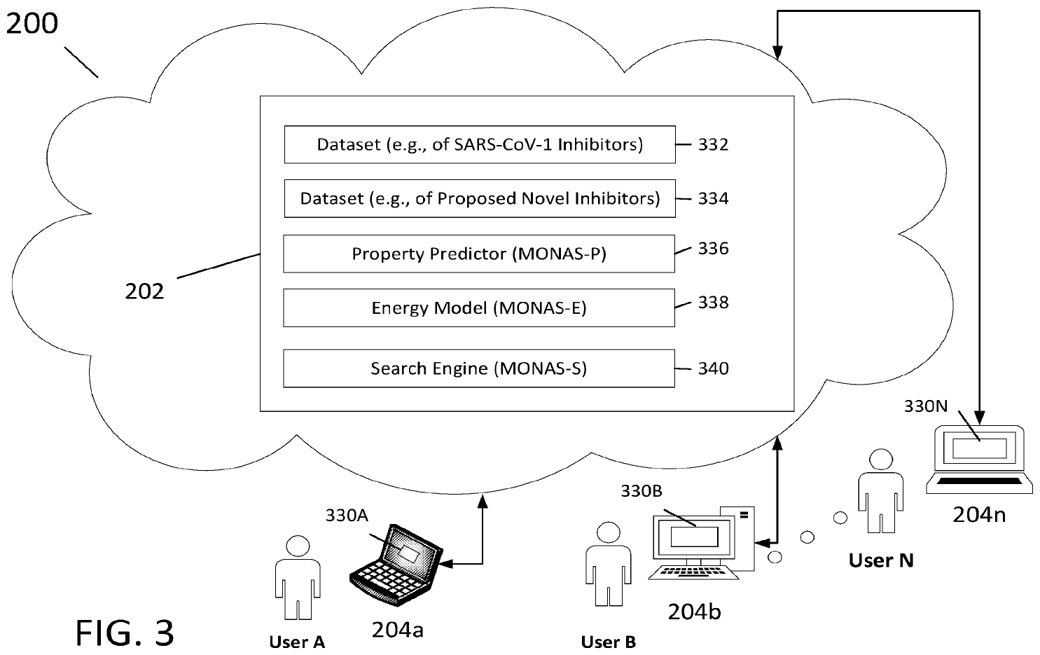

In some implementations, each client 204a, 204b, . . . 204n may be configured to perform one or more of the design functionalities disclosed herein without requiring involvement of the server 202. In some implementations, the design functionalities disclosed herein may be distributed between the server 202 and the clients 204a, 204b . . . 204n. In some implementations, a significant portion (or all) of the design functionalities disclosed herein may be performed by the server 202, with the client devices 204a, 204b . . . 204n merely serving as user interface terminals. Various implementations may include one or more servers 202. In implementations that have more than one server, the servers 202 may collaborate with one another and/or with one or more of the clients 204a, 204b . . . 204n to provide or perform the design functionalities disclosed herein. Moreover, in a typical implementation, the clients 204a, 204b . . . 204n enable human users to access and/or interact with the systems and techniques disclosed herein (e.g., to enter data, initiate design functionalities, view or otherwise access results, etc.) FIG. 3 is a more detailed schematic representation showing an implementation of the network environment 200 represented in FIG. 2.

The network environment 200 in FIG. 3 has a computer-based platform for designing novel potential inhibitors. The platform, in the illustrated implementation, is deployed on server 202 and is accessible for utilization by users (User A, User B . . . User N) from any one or more of a plurality of network-connected computer-based user workstations 204a. 204b . . . 204n. The components of the platform in the illustrated implementation include a dataset of SARS-CoV-1 inhibitors 332, a dataset of proposed novel inhibitors 334, an inhibition predictor (MONAS-P) 336, an energy model (MONAS-E) 336, and a search engine (MONAS-S) 338. In a typical implementation, each item of data from the dataset is a representation of a molecule as represented in the figures. There are a number of ways a system might display individual molecules, e.g., graphically or as their representative SMILES strings. In a typical implementation, the dataset of SARS-CoV-1 inhibitors 332, and the dataset of proposed novel inhibitors 334 are stored in computer-readable medium, such as memory 104 or storage device 106. In various implementations, any one or more (or all) of the other platform components (e.g., the inhibition predictor 336, the energy model 336, and/or the directed search engine 338) may be implemented utilizing hardware or a combination of computer hardware and software (e.g., in the form of a computer-based processor executing associated computer-readable instructions stored on a computer-readable medium).

Each workstation includes whatever hardware and software may be required to implement and provide a virtual environment 330a, 330b . . . 330n within which users can access design functionalities described herein and/or results produced by the computer-based functionalities disclosed herein.

In general terms, and in various implementations, the computer system 100 in FIG. 1 and/or the network environment 200 represented in FIGS. 2 and 3 is particularly well-suited to design/identify novel potential inhibitor molecules quickly, efficiently and accurately. This is important because, in recent history, the search for molecules which may inhibit key receptor sites of Severe Acute Respiratory Syndrome Coronavirus-2 (SARS-CoV-2) has emerged as a central research objective within the scientific community. The systems and techniques disclosed herein, in a typical implementation, deploy the use of Deep Learning (DL) techniques to this task. At present, there are existing datasets of SARS-CoV-1 inhibitors, but there is a comparable lack of large-scale data for SARS-CoV-2 inhibition. That said, there are a number of structural similarities between both the main 3CL^{P}? protease and the PL^{P}? protease of SARS-Cov-2, both used in reproduction, and those of Severe Acute Respiratory Syndrome Coronavirus-1 (SARS-CoV-1). Based on this, it is suggested that the lack of available large-scale data for SARS-Cov-2 inhibition can be overcome, at least somewhat, by using and leveraging existing datasets of SARS-CoV-1 inhibitors to identify not only other novel potential SARS-CoV-1 inhibitors, but also novel potential SARS-CoV-2 inhibitors, as disclosed herein.

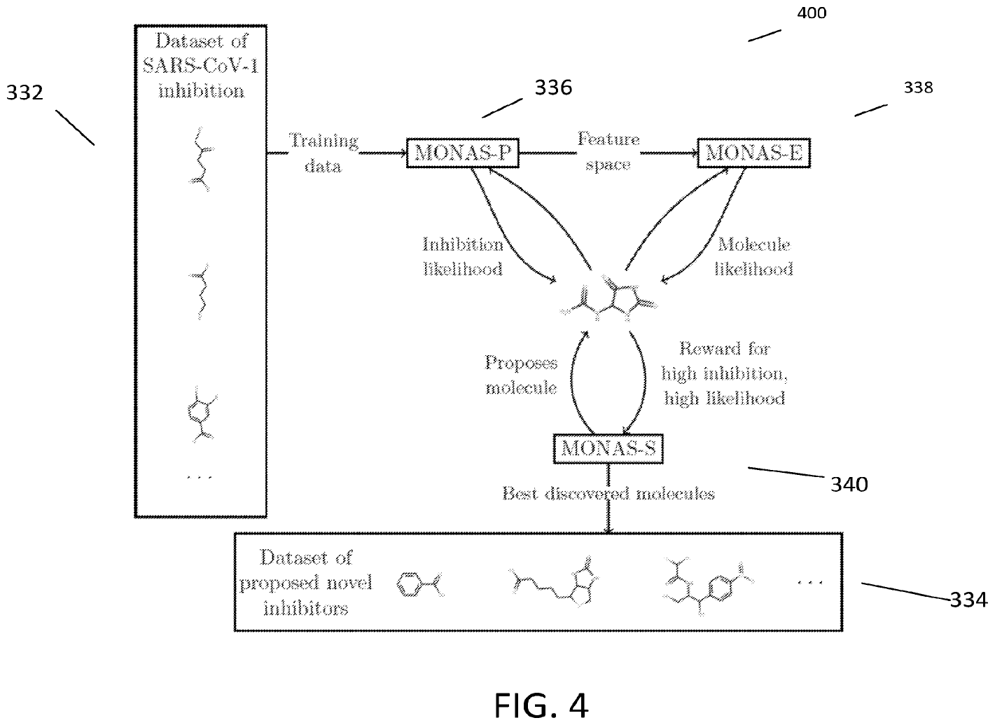

FIG. 4 is a schematic representation showing components of an exemplary design platform 400 from the network 200 in FIG. 3 as well as dataflow between those components according to one exemplary embodiment. The illustrated schematic represents a general data-driven automatic molecule design framework and approach that involves the three core functional components mentioned above: the property predictor 336 (MONAS-P) 336, the energy model 338 (MONAS-E), and the search engine 340 (MONAS-S). The exemplary design platform 400 in FIG. 4 is particularly configured and operable to design novel SARS-CoV-1 inhibitor molecules. In addition to the three core functional components just mentioned, FIG. 4 also shows a dataset of molecules already identified as having SARS-CoV-1 inhibition properties (i.e., one or more datasets of SARS-CoV-1 inhibition 332) and a dataset of proposed inhibitor molecules designed by the illustrated system 338 (i.e., a dataset of proposed novel inhibitors 334).

The property predictor (MONAS-P) 336 is generally configured to predict the likelihood of particular molecules having particular properties. More specifically, in the illustrated implementation, the property predictor (MONAS-P) 336 is a machine-learning model, trained (e.g., on one or more existing datasets) and thereby configured to predict the likelihood of a particular molecule having a particular property (e.g., being an inhibitor). The property predictor (MONAS-P) 336 can be implemented as a predictive model of molecular activity and used as a guide to identify new molecules with one or more desirable properties. In other implementations, the property predictor (MONAS-P) 336 could use Long Short-Term Memory (“LSTM”) to predict molecular behavior based on SMILES (i.e., “simplified molecular-input line-entry system”) strings for the molecules. In an exemplary implementation, the inhibition predictor 336 is implemented as a computer-based deep neural network (e.g., a graph-neural network, GNN) trained on training data from the dataset of SARS-CoV-1 inhibition 332 to predict inhibition of SARS-CoV-1 proteases.

The energy model (MONAS-E) 338 is configured to assign a measure of statistical similarity between proposed molecules (e.g., from MONAS-S) and the universe of molecules represented in the training data (e.g., molecules represented in the dataset of SARS-CoV-1 inhibition molecules 332). This measure is referred to in FIG. 4 as a molecule likelihood. In a typical implementation, the energy model (MONAS-E) performs this function by assigning a scalar (representing an energy) to each proposed molecule that represents the measure of statistical similarity between that proposed molecules and the universe of molecules represented in the training data. In some implementations, the energy model (MONAS-E) is implemented as a general probabilistic model that is trained (i.e., learns) to model the distribution of molecules (e.g., across the universe of molecules represented in the training data) in an unsupervised manner. In some implementations, the energy model (MONAS-E) 338 may use Deep Energy Estimator Networks (“DEEN”) to perform its functionalities. In other implementations, DEEN could be replaced with a variational autoencoder.

The search engine (MONAS-S) 340 is configured to perform a (potentially multi-objective) search procedure that explores the space of possible molecules guided by the property predictor (MONAS-P) 336 and the energy model (MONAS-E) 338 to discover the best molecules for SARS-CoV-1 inhibition. According to the illustrated implementation, the search engine (MONAS-S) 340 is configured to propose molecules for consideration in this regard. For each proposed molecule, the property predictor (MONAS-P) 336 assigns a value representing inhibition likelihood to the molecule, and, for each proposed molecule, the energy model (MONAS-E) assigns a value representing a statistical similarity between the proposed molecule and the universe of molecules represented in the training data (i.e., a molecule likelihood) to the molecule. The search engine (MONAS-S) 340 utilizes this information to discover the best possible molecules (i.e., those molecules having the most favorable combination of inhibition likelihood and statistical similarity to molecules already identified as having SARS-CoV-1 inhibition properties. In essence, and as represented in FIG. 4, those molecules (having, e.g., high inhibition likelihood and high molecular similarity to known SARS-CoV-1 inhibitor molecules), are rewarded and end up being placed into the dataset of proposed novel inhibitors 334. In a typical implementation, the molecules that end up having relatively low scores of inhibition likelihood and/or molecular similarity may be discarded (i.e., not saved into the dataset of proposed novel inhibitors 334).

Once a particular proposed molecule has been assessed (and, e.g., either stored in the dataset of proposed novel inhibitors 334 or not), the search engine (MONAS-S) 340 proposes another molecule for consideration in this regard. The search engine's selection in this regard may be guided or influenced by the feedback provided on inhibition likelihood and molecular similarity from the property predictor (MONAS-P) 336 and the energy model (MONAS-E) 338 for one or more prior proposed molecules. Thus, in such implementations, the search engine's selection of molecules to propose for consideration can be guided (or directed) by the outcome of previous assessments of other molecules. In some implementations, the search engine (MONAS-S) 340 utilizes Monte Carlo Tree Search (“MCTS”) to search over derivations of molecules in a Backus-Naur form grammar. In other implementations, MCTS could be replaced with genetic algorithm(s) or deep reinforcement learning. The exact degree of likelihood of inhibition and similarity to known molecules that would warrant the directed search engine flagging a proposed molecule as one of the best of the proposed new molecules can vary and, in some instances, may depend on the distribution of characteristics of the set of proposed molecules itself.

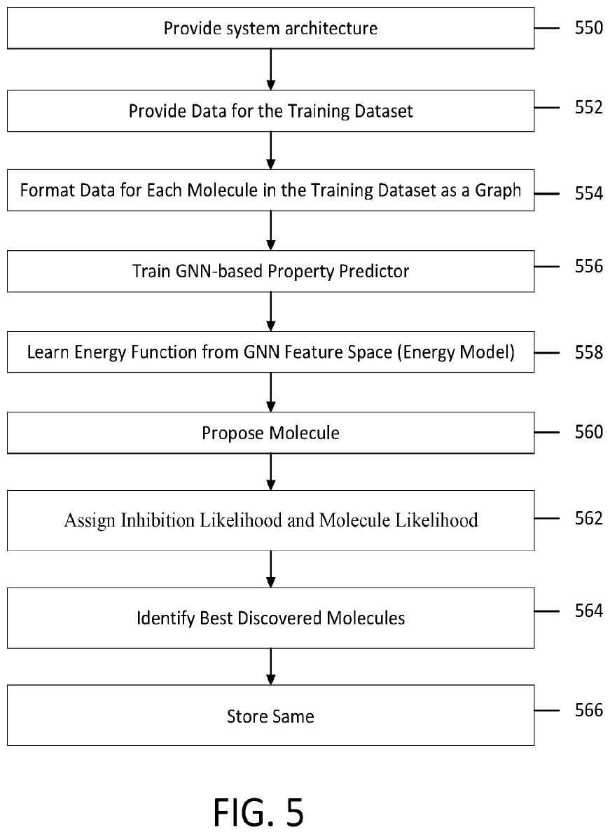

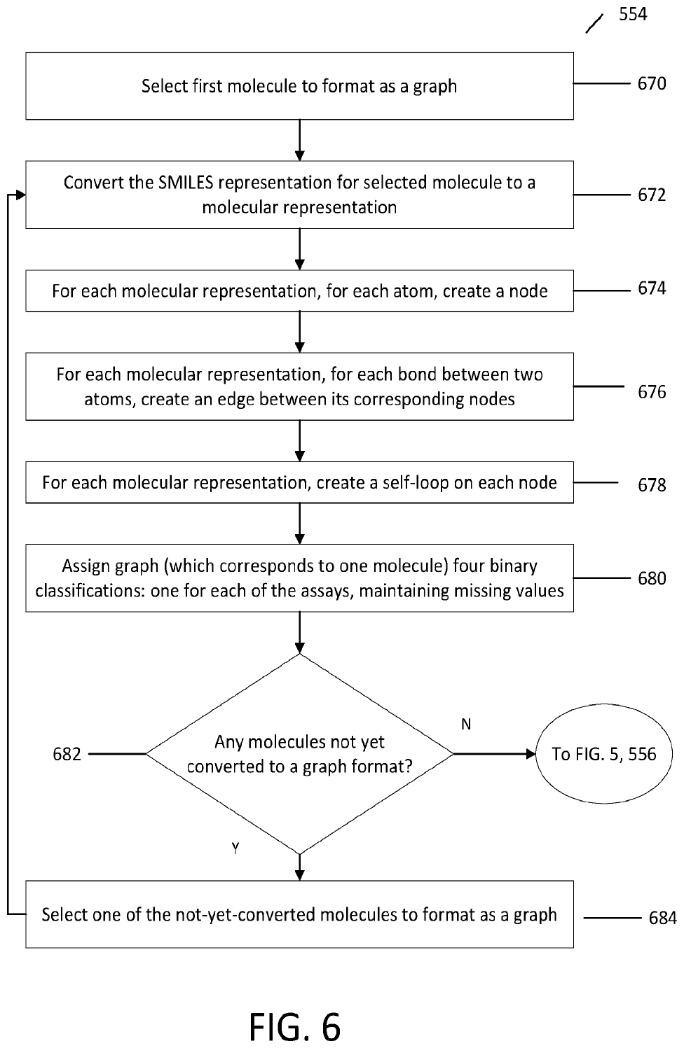

FIG. 5 is a flowchart that represents an exemplary process of designing novel 3CL^{pro} and/or PL^{pro} inhibitors that may be implemented, for example, utilizing the system represented in FIGS. 1 through 4.

According to the illustrated flowchart, the process begins (at 550) by providing the basic system architecture to perform the subsequent process steps.

In a typical implementation, this step 550 may include configuring and/or providing a computer system or network (“system”) like the one shown in FIGS. 1-4. The system, in a typical implementation, includes one or more computer-based processors (e.g., 102) and computer-readable media 104, 106—volatile and/or non-volatile memory-loaded with software (i.e., computer-readable instructions that, when executed by the computer-based processor(s), 102 cause the computer-based processor(s) 102 to perform the computer-based functionalities disclosed herein). The software and computer-based processor(s) 102, in various implementations, are configured to implement the property predictor (MONAS-P) 336, the energy model (MONAS-E) 338, and the search engine (MONAS-S) 340. Additionally, in a typical implementation, the computer-readable media 104, 106 stores data to support the various computer-based functionalities disclosed herein. The data typically is organized into two datasets—the dataset of SARS-CoV-1 inhibition 332 and the dataset of proposed novel inhibitors 334.

Next, the illustrated method (at 552) includes providing data for the SARS-CoV-1 inhibition dataset 332. The data for the SARS-CoV-1 inhibition dataset 332 may come from any one or more of a variety of different data sources and may include any one of a variety of different types of data. In one exemplary implementation, the SARS-CoV-1 inhibition dataset 332 is populated with data from fours assays in PubChem (see, e.g., Sunghwan Kim, Jie Chen, Tiejun Cheng, Asta Gindulyte, Jia He, Siqian He, Qingliang Li, Benjamin A Shoemaker, Paul A Thiessen, Bo Yu, et al. Pubchem 2019 update: improved access to chemical data. Nucleic acids research, 47(D1):D1102-D1109, 2019). One example of these assays is summarized in Table 1.

TABLE 1

| Assay ID | Target protease | #Inactive molecules | #Active molecules |
|---|---|---|---|
| 1,706 | 3CL^{pro} | 290,321 | 405 |
| 1,879 | 3CL^{pro} | 244 | 136 |
| 485,353 | PL^{pro} | 322,433 | 602 |
| 652,038 | PL^{pro} | 735 | 198 |
| Total | — | 331,480 | 1,095 |

For each assay, Table 1 identifies an assay ID (e.g., identifying numbers for an “assay”). For each assay ID, Table 1 identifies a target protease. According to Table 1, molecules associated with assay ID 1,706 and assay ID 1,879 are known to target the 3CL^{pro} protease. Molecules associated with assay ID 485,353 and assay ID 652,038 are known to target the PL^{pro} protease. Table I also identifies, for each assay ID, the number of inactive and the number of active molecules. Each molecule in each assay may (active) or may not (inactive) inhibit the given protease. These columns in the table simply indicate how many cases of each of these two scenarios there are. So, for example, in assay 1,706, there are 290,321 molecules which do not inhibit the 3CL^{pro} protease, and there are 405 molecules which do inhibit the 3CL^{pro} protease.

The calculation of totals (at the bottom of Table 1) accounts for the fact that the same molecule may appear in multiple different assays. Therefore, for example, the total number of active molecules represents the number of molecules that are active in at least one assay. Likewise, the total number of inactive molecules represents the number of molecules that are inactive in at least one assay.

Each assay represents a set of molecules (represented by SMILES strings) and test results indicating a shared characteristic among the set of molecules (e.g., ability to inhibit a given protease for SARS-CoV-2, etc.). Each SMILES string has a series of characters and/or symbols that represent the chemical structures and characteristic(s) of the associated molecule in a manner that can be used by a computer. For example, in an exemplary SMILES string, an atom may be represented by its atomic symbol, bonds may be denoted with different symbols depending on bond type (or by the absence of symbol), chain structures may be represented by combining atomic symbols and bond symbols, a branch from a particular chain structure may be specified by placing any SMILES symbols for the branch between parentheses, ring structures may be represented by numbers to identify the opening and closing ring atoms, etc. Various molecular characteristics (e.g., ability to inhibit), as determined through testing, can be expressed in a SMILES format, too. In some instances, for example, each assay can be viewed as a mapping of a set of molecules MA to a binary vector, e.g., {0, 1}, where 0 indicates no inhibition and 1 indicates inhibition. In a typical implementation, the system 100 is configured to interpret and utilize assay data represented in a computer-based environment in this manner.

Next, according to the illustrated implementation, the system 100 (at 554) formats the assay data for each molecule as a graph in order to facilitate using the assays with a graph neural network (e.g., the inhibition predictor 336). There are a variety of ways that this formatting step may be untaken.

FIG. 6 is a flowchart representing one exemplary implementation of a process for formatting the data representing the various molecules in the assays as graphs.

According to the process represented in the illustrated flowchart, the system 100 (at 670) selects a first molecule from the assays to format as a graph. There are a variety of ways in which this initial selection may be performed. In various implementations, for example, the selection may be random, the selection may be based on the order that the molecular data appears in the assays, the selection may be performed based on some previously-learned intelligence, etc. In essence, however, during the selection process, the system 100 identifies the data for a particular one of the molecules from the datasets in the assays.

Next, according to the process represented in the illustrated flowchart, the system 100 (at 672) converts the SMILES representation for the selected molecule to a molecular representation (e.g., the graph representation described in the flowchart). There are a variety of ways in which this step may be accomplished. According to one exemplary implementation, the SMILES representations are converted to molecular representations using RdKit. Rdkit is an open-source, computer-based cheminformatics toolkit. More specifically, RdKit is a software development kit that allows user to develop custom computer applications for use in virtual screening, chemical database mining, structure-activity studies, structural comparisons, etc. (See, e.g., Greg Landrum: RdKit: Open-Source cheminformatics at the RdKit website, www.redkit.org).

Next (at 674), for each atom in the molecular representation for the selected molecular structure, the system 100 creates a node in a computer-based graph data structure. In general, a graph data structure typically consists of a set of nodes (also called vertices or points) connected by edges (also called links or lines). In an exemplary implementation, the node that the system 100 creates represents (and has stored in association with it) one or more (or all) of the following features of the associated atom: mass, valence, the total number of hydrogens, whether it is aromatic, formal charge, etc. Generally speaking, hydrogen atoms may only form single bonds with a single atom and therefore do not contribute anything structural to the molecule. As a result, they are typically not directly represented as atoms but instead all other atoms are labelled with how many hydrogen atoms are attached to them.

Next (at 676), for each bond between two atoms in the molecular representation for the selected molecular structure, the system 100 creates an edge that extends in each direction between the corresponding nodes in the computer-based graph data structure. The edge typically extends in each direction between the corresponding nodes. The system 100, in a typical implementation, categorizes each edge as corresponding to one of: a single bond, a double bond, a triple bond, an aromatic bond, etc.

Next (at 678), for each bond between two atoms in the molecular representation for the selected molecular structure, the system 100 creates a self-loop on each node. In a computer-based graph, a self-loop is an edge that connects a node to itself. This helps to ensure that each node is treated as a member of its own neighborhood within the GNN. A GNN generally updates each node/atom/vertex's values based on the values on the nodes that are adjacent to it e.g., connected by an edge. These adjacent nodes are generally referred to as the node's “neighborhood.” By creating an edge from a node to itself (e.g., a loop), a node is now treated as a member of its own neighborhood, which means that the node's own value can then condition the computation drawn from the neighborhood. This does not generally change the underlying abstract functionality of the computer-based model at all.

Next (at 680), the system 100 assigns the graph (which represents one molecule) four binary classifications; one for each of the four assays, maintaining missing values. For each assay, a molecule may either be labeled ‘inactive’ (does not inhibit the protease), ‘active’ (does inhibit the protease), or may not be labeled at all (missing value). If, for example, we let ‘0’ represent inactive, ‘1’ represent active, and ‘?’ represent missing. Then for a molecule which is ‘active’ in assay 1,706, ‘inactive’ in assays 1,879 and 485,353 and not labelled at all in assay 652,038. Then its four binary values (maintaining the missing unlabeled value) are 1, 0, 0, ?.

Next (at 682), the system 100 determines whether there are any molecules represented in the assays whose SMILES string has not yet been converted to a graph. If so, the system 100 (at 684) selects one of the not-yet-converted molecules for conversion. And the process returns to step 672. If the system 100 determines that there are no further molecules represented in the assays whose SMILES string has not yet been converted to a graph, the system 100 proceeds to step 556 in FIG. 5.

Step 556 in FIG. 5 involves training the GNN-based property predictor (MONAS-P) 336, which, in the illustrated implementation, is an inhibition predictor. There are a variety of methods for performing this step, some of which are disclosed herein. In an exemplary implementation, the property predictor (MONAS-P) is trained in a supervised manner to predict one or more desirable properties of molecules, using the training dataset(s). In principle this can be to minimize any supervised loss. In the case of numerical properties, typical cases are Mean Square Error (MSE) or its variants, such as Normalized Root Mean Square Error (NRMSE). In the case of categorical properties, typical loss is Cross Entropy. It is generally good practice to reserve a “validation” set, a subset of the training data used to determine when the model is overfitting.

Next (at 558), the method represented in the illustrated flowchart includes learning an energy function, with the energy model (MONAS-E) 338, from the feature space of the property predictor (MONAS-P) 336. There are a variety of methods for performing this step, some of which are disclosed herein. In a typical implementation, the energy model (MONAS-E) 338 is trained in an unsupervised manner to model the distribution of molecules in the training dataset(s).

Next (at 560), the method represented in the illustrated flowchart includes proposing a molecule (e.g., a molecule to be considered as a novel inhibitor candidate). There are a variety of methods for performing this step. In a typical implementation, however, the step is performed by the search engine (MONAS-S) 340. Moreover, as disclosed herein, this step may relate to searching performed by the search engine (MONAS-S) 340 to explore the space of possible molecules guided by the property predictor (MONAS-P) 336 and the energy model (MONAS-E) 338 to discover the best molecules for SARS-CoV-1 inhibition. In a typical implementation, the search engine searches for molecules which maximize the likelihood and/or magnitude of one or more desired properties, according to the property predictor, while also maximizing the likelihood of proposed molecules also belonging to (or being highly similar to) the molecules of the original training dataset(s). One way to do this that is disclosed herein—the predicted properties can be multiplied by a [0,1] value computed from the energy function. Then the highest scoring molecules have high property predictions and high statistical likelihood.

Next (at 562), the method represented in the illustrated flowchart includes assigning an inhibition likelihood and a molecule likelihood (i.e., a measure of statistical similarity between the proposed molecules and the universe of molecules represented in the training dataset). There are a variety of ways in which this step can be accomplished. In a typical implementation, however, the property predictor (MONAS-P) 336 is involved in assigning the inhibition likelihood and the energy model (MONAS-E) is involved in assigning the molecule likelihood.

Next (at 564), the method represented in the illustrated flowchart includes identifying the best discovered molecules from among proposed molecules. There are a variety of ways in which this step might be undertaken. However, in a typical implementation, it is a step undertaken by the search engine (MONAS-S) 340. In some implementations, the search engine (MONAS-S) 340 may, for example, rank all of the proposed molecules that have been assigned an inhibition likelihood and a molecule likelihood (in step 562) based on some combination (e.g., weighted or otherwise) of the values assigned for those likelihood to produce a combined score. In such implementations, the best among those proposed molecules may simply be those molecules that received a high combined score. In some such implementations, the process may involve some minimum combined score for inclusion in the best molecules list. In some implementations, the process may involve including only those molecules having a combined score that places them in the top x % (where x is a number, e.g., 50%, 40%, 30%, 20%, 10%, etc.) In some such implementations, the process may involve other methods for identifying the best molecules. However, in each method, the best molecule(s) is(are) identified based, at least in part, on the assigned inhibition (or, more generically, property) likelihood, the molecule likelihood, or some combination thereof.

Next (at 566), the process represented in the illustrated flowchart includes storing data identifying (and, optionally, related to) the identified best molecules. In a typical implementation, this information is stored as a dataset of proposed novel inhibitors (see, e.g., 334 in FIGS. 3 and 4) in one or more of the computer-based memory components (e.g., 104, 106 in FIG. 1). In some implementations, the molecule identifiers are labeled with numerical or categorical information describing desirable properties such as inhibition likelihood, molecule likelihood, efficacy or activity with respect to a medical function, toxicity, drug likeness, synthetic accessibility, etc.

Once the so-called best molecules are stored in the dataset of proposed novel inhibitors (at 566 in FIG. 5), those molecules may undergo further testing and evaluation for possible usefulness in inhibiting SARS-CoV-1 and/or used (i.e., administered to people) to inhibit the same.

### Graph Neural Networks (GNNs)

In a typical implementation, the property predictor (MONAS-P) 336, for example, is a graph neural network. Graph neural networks are a relatively recent form of deep learning architecture for reasoning about structured relational data. As molecules are naturally structured, with atoms as nodes and bonds as edges, there is a clear affinity between graph neural networks and predictive molecular chemistry. In an exemplary implementation, the property predictor (MONAS-P) 336, for example, is instantiated as a GNN architecture.

### GNN Layers

While there are a variety of unique GNN designs that could be applied for use (e.g., as the property predictor (MONAS-P) 336), a typical construction comprises a layer which takes a directed labelled multi-graph G_{K}=(V_{K}, E_{K}) and computes new node and edge features based on the existing features and adjacency, yielding a new graph G_{K+1}=(V_{K+1}, E_{K+1}) with the same structure but new labels. Here, the set V_{K}={vᵢ}_{i=1:Nv} is the set of Nv nodes where each vᵢ is the ith node's features, and E_{K}={eⱼ, sⱼ, tⱼ}_{j=1:Ne} is the set of Ne edges where each eⱼ is the jth edge's features, sⱼ∈V_{K} is the jth edge's source node and tⱼ∈V_{K} is the jth edge's target node. The transformed G_{K+1} can then be passed to another graph neural network layer, have its node features used to classify nodes (see, e.g., Thomas N. Kipf and Max Welling. Semi-supervised classification with graph convolutional networks. in International Conference on Learning Representations, 2017; and Bryan Perozzi, Rami Al-Rfou, and Steven Skiena. Deepwalk: Online learning of social representations. in Proc. 20th ACM SIGKDD International Conference on Knowledge Discovery and Data Mining, page 701-710. Association for Computing Machinery, 2014.), or predict edges (see, e.g., Thomas Kipf, Ethan Fetaya, Kuan-Chieh Wang, Max Welling, and Richard Zemel. Neural relational inference for interacting systems. arXiv preprint ar Xiv:1802.04687, 2018; and Anh-Tien Ton, Francesco Gentile, Michael Hsing, Fuqiang Ban, and Artem Cherkasov. Rapid identification of potential inhibitors of sars-cov-2 main protease by deep docking of 1.3 billion compounds. Molecular informatics, 2020.), or features can be pooled and passed to some other neural network to perform classification or regression on the entire input graph (see, e.g., David K Duvenaud, Dougal Maclaurin, Jorge Iparraguirre, Rafael Bombarell, Timothy Hirzel, Alan Aspuru-Guzik, and Ryan P Adams. Convolutional networks on graphs for learning molecular fingerprints. In Advances in Neural Information Processing Systems, volume 28, pages 2224-2232, 2015; and Zhen Zhang, Jiajun Bu, Martin Ester, Jianfeng Zhang, Chengwei Yao, Zhi Yu, and Can Wang. Hierarchical graph pooling with structure learning. arXiv preprint arXiv:1911.05954, 2019.)

In the following it will be helpful to refer to the neighborhood of a given node vᵢ, e.g. the set of edges which target it. This is defined by nᵢ={j|eⱼ∈E_{K} and tⱼ=vᵢ}. In this document, a simple model of graph neural network layers of a form (e.g., described in Peter W Battaglia, Jessica B Hamrick, Victor Bapst, Alvaro Sanchez-Gonzalez, Vinicius Zambaldi, Mateusz Malinowski, Andrea Tacchetti, David Raposo, Adam Santoro, Ryan Faulkner, et al. Relational inductive biases, deep learning, and graph networks. arXiv preprint arXiv:1806.01261, 2018) may be used. In an implementation, to compute G_{K+1} from G_{K}, the system 100 first computes E_{K+1}, given by E_{K}={eⱼ′, sⱼ, tⱼ}_{j=1:Ne} where eⱼ″ is a function of each edge's features, source node features and target node features: eⱼ′=Φ_{E}(eⱼ, sⱼ, tⱼ) where Φ_{E} is a multi-layer perceptron (MLP). Then the system 100 computes the mean of the new edge features of the neighborhood of each node vᵢ as,

$${\overline{𝒩}}_{i} = \frac{1}{\vert {𝒩}_{i} \vert} {\sum }_{j ∈ {𝒩}_{i}} {e}_{j}^{′} . \qquad ( 1 )$$

The system 100 then computes new node features V_{K+1} as a function of each node's features and its mean aggregated neighborhood,

V_{K+1}={vᵢ′}_{i=1:Nv} (2)

Vᵢ′=Φ_{V}(vᵢ,nᵢ) (3)

giving the updated graph G_{K+1}. As before, Φ_{V} is a multi-layer perceptron. An important property of the above construction is that, because each update is in the context of a node or edge's adjacency, and the permutation invariant mean aggregation of the neighborhood is used, the entire layer is invariant to permutations of the node and edge sets. Therefore, two isomorphic graphs will be updated in the same manner irrespective of the order in which the nodes (and edges) are indexed.

When classifying graphs, it is often helpful to ‘coarsen’ the graph by merging nodes (see, e.g., Jie Zhou, Ganqu Cui, Zhengyan Zhang, Cheng Yang, Zhiyuan Liu, Lifeng Wang, Changcheng Li, and Maosong Sun. Graph neural networks: A review of methods and applications. arXiv preprint arXiv:1812.08434, 2018) to reduce the number of parameters and avoid over-fitting. In some implementations, the edge contraction pooling operator EdgePool (see, e.g., Frederik Diehl. Edge contraction pooling for graph neural networks. arXivpreprint arXiv:1905.10990, 2019) is used to achieve this effect, which is extended to support edge features. EdgePool allows the graph coarsening to be learned through parameters Wand b, such that a raw score can be assigned (by system 100) to each edge eⱼ from source node sⱼ to target node tⱼ as,

r(eⱼ)=W(eⱼ⊕sⱼ⊕tⱼ)+b (4)

where Φ denotes concatenation. This is transformed into a score value s(eⱼ) by taking the softmax of the neighborhood of source node:

s(eⱼ)=softmaxₙ_{ᵢ}r(eⱼ) (5)

The system 100 then iteratively contracts the edges according to their score. That is, starting at the edge with the highest score and continuing in descending order, the system 100 contracts an edge if and only if its source and target nodes have not been involved in any other edge contraction. When an edge eⱼ is contracted, it is removed, its source node sⱼ and its target node tⱼ are merged (by the system 100), replacing all 3 items with a single node with the average of the node features multiplied by the score edge's associated score:

$${{v}_{j}}^{′} = s ( {e}_{j} ) \frac{{s}_{j} + {t}_{j}}{2} \qquad ( 6 )$$

Any incoming or outgoing edges of either sⱼ or tⱼ now are instead connected to vⱼ′. Furthermore, any edges which have become parallel as a result of this merge have their features merged, again by averaging. The result of these steps is a process whereby the graph is coarsened in a learned fashion while connectivity is preserved. In contrast, in alternative learnable pooling approaches such as Top-K pooling (see, e.g., C\check{a}talina Cangea, Petar Veličković, Nikola Jovanović, Thomas Kipf, and Pietro Liò. Towards sparse hierarchical graph classifiers. arXiv preprint arXiv:1811.01287, 2018) and self-attention graph pooling (see, e.g., Junhyun Lee, Inyeop Lee, and Jaewoo Kang. Self-attention graph pooling. arXivpreprint arXiv:1904.08082, 2019), the fact that nodes are dropped, rather than merged, means that connectivity is lost.

### Property Predictor (MONAS-P) Architecture

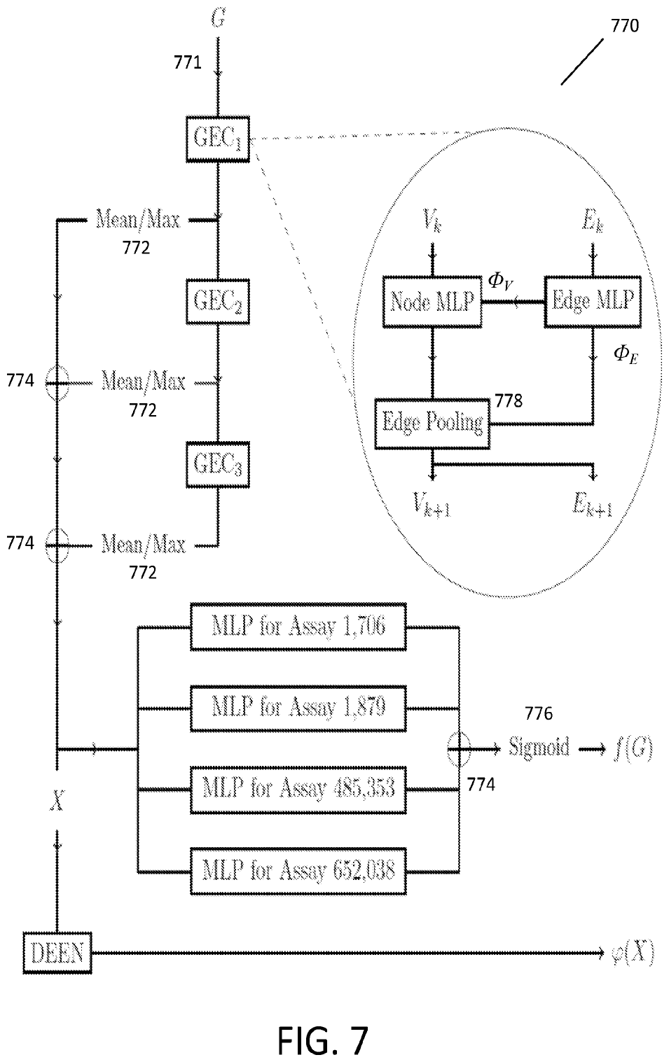

In some implementations, the system 100 is configured to treat prediction of inhibition as multi-task and has a single network which predicts inhibition for all four assays. An exemplary computer-based architecture 770 to perform this functionality is shown in FIG. 7.

The exemplary architecture represented in FIG. 7 has an input 771, “GNN Layer-Edge Contraction” blocks (GEC1, GEC2, GEC2), global mean and global max pooling elements 772, concatenators 774, a multi-layer perceptron (MLP) for each assay, and a sigmoid function operator 776. Each GEC block is a graph neural network layer, consisting of a node MLP Φ_{V} and an edge MLP Φ_{E}, followed by an edge contraction pooling operator 778.

Initially, an input graph G is passed, via the input 772, through the three “GNN Layer-Edge Contraction” (GEC) blocks (GEC1, GEC2, GEC3). In an exemplary implementation, in every GEC block, both the node MLP Φ_{V} and the edge MLP Φ_{E} are 2-layer MLPs with 96 neurons per layer, and batch normalization is applied after each layer followed by rectified linear unit (ReLU) activation. Each edge contraction pooling operator 778 removes approximately half of the edges from the graph so that the graph, after the third GEC block (GEC3), is expected to have ⅛th of the edges of the input graph G.

After each GEC block (GEC1, GEC2, GEC3), global mean and global max pooling are applied (at 772) to the 96-dimensional node features, yielding a 192-dimensional representation of the input. The three representations from the GEC blocks are concatenated together (at the 774s), yielding the 574-dimensional latent representation of the input, denoted X.

The latent representation X is then passed to the Deep Energy Estimator Network (DEEN) of energy model (MONAS-E) to generate an energy p(X).

For each assay, a “head” is used, which, in the illustrated implementation, is a specialist MLP that predicts only inhibition for that assay. A head is a name of a sub-component of a neural network which performs and outputs a specific function. For example, a network can perform functions A and B. While most of the network may perform computations for both tasks, a small sub-section of the network may perform computation specific to function A only, and outputs for function A only. This sub-section, for example, may be referred to as a ‘head’.

Each head is provided with the latent representation X and, in an exemplary implementation, applies two 128-neuron feed-forward layers, with dropout applied before each layer (p=0.25 and p=0.5, respectively), and batch normalization and ReLU activation after. Then, the 128-dimensional vector is passed to a single logit neuron which gives a prediction for the given assay. The predictions are concatenated and passed through a softmax (or sigmoid) operator, yielding the prediction vector ƒ(G).

### Inhibition Loss

In an exemplary implementation, to handle the multitask context while maintaining efficiency, the collective 331,480 molecules are treated as a single dataset and are passed through the GNN in a mini-batch fashion. However, adjustments may be made to a standard binary cross-entropy loss function to ensure that the network is not biased towards any given assay and that missing data is appropriately handled. For a given graph (molecule) G with ground truth K_{A} for assay A, the loss is computed for the A-th head of the model, ƒ_{A}(G), as

$$ℓ ( {K}_{A} , {J}_{A} ( G ) ) = \{ \begin{matrix}- {α}_{A} {β}_{A} log ( {J}_{A} ( G ) ) , & {K}_{A} = 1 \\ - {α}_{A} log ( 1 - {J}_{A} ( G ) ) , & {K}_{A} = 0 \\ 0 , & {K}_{A} is missing ,\end{matrix} , \qquad ( 7 )$$

where the loss for one single molecule/graph across all assays is given by

$$ℓ ( K , G ) = {\sum }_{A} ℓ ( {K}_{A} , {f}_{A} ( G ) ) , \qquad ( 8 )$$

and the total loss l(Θ) to be minimized,

$$ℓ ( θ ) = {𝔼}_{( K , G )} ℓ ( K , G ) , \qquad ( 9 )$$

The factors α_{A} and β_{A} are weights towards each task and positive instances of that task, respectively to handle the extreme imbalances of the data (see Table 1), for example. For assay A with I training inhibitors, J training non-inhibitors and N total training samples, α_{A}=N/(I+J) and, β_{A}=(I+J)/I. The role of α_{A}>1 is to ensure that each assay contributes equally to the total loss averaged across all N training samples, whereas β_{A}>1 ensures that positive and negative samples for each assay contribute to the total loss averaged across all I+J training molecules for assay A.

### MONAS-E: Deep Energy Estimator Network (DEEN)

Constructing a probabilistic model for the molecules and a statistical measure of how similar two molecules are based on dataset samples faced two main challenges: (i) In high dimensions, classical techniques like kernel density estimation break down because the volume of space grows exponentially (i.e., the curse of dimensionality) and nonparametric methods which are based on Euclidean similarity metric become inadequate. (ii) One typically resorts to parameterizing distributions in directed or undirected graphical models, but, in both cases, the inference over latent variables is in general intractable. At the root of the problem lies in estimating the normalizing constant (a.k.a. the partition function) of probability distributions in high dimensions.

There is however a class of models, called energy models, that are formulated around learning unnormalized densities or energy functions. For Monte Carlo Tree Search (“MCTS”), where only the likelihood of generated molecules relative to the likelihood of the molecules in the dataset is of importance (this will become clearer in the next section), the distribution needs only be known up to a constant. Therefore, learning energy functions is sufficient. In this section, this is based on recent developments (see, e.g., Saeed Saremi and Aapo Hyvarinen. Neural empirical Bayes. Journal of Machine Learning Research, 20: 1-23, 2019) that formulate the problem of learning energy functions in terms of the empirical Bayes methodology (see, e.g., Koichi Miyasawa. An empirical Bayes estimator of the mean of a normal population. Bulletin of the International Statistical Institute, 38 (4): 181-188, 1961; and Herbert Robbins. An empirical Bayes approach to statistics. In Proc. Third Berkeley Symp., volume 1, pages 157-163, 1956.) The framework is referred to by “DEEN” due to its origin in Deep Energy Estimator Networks (see, e.g., Saeed Saremi, Arash Mehrjou, Bernhard Schölkopf, and Aapo Hyvarinen.

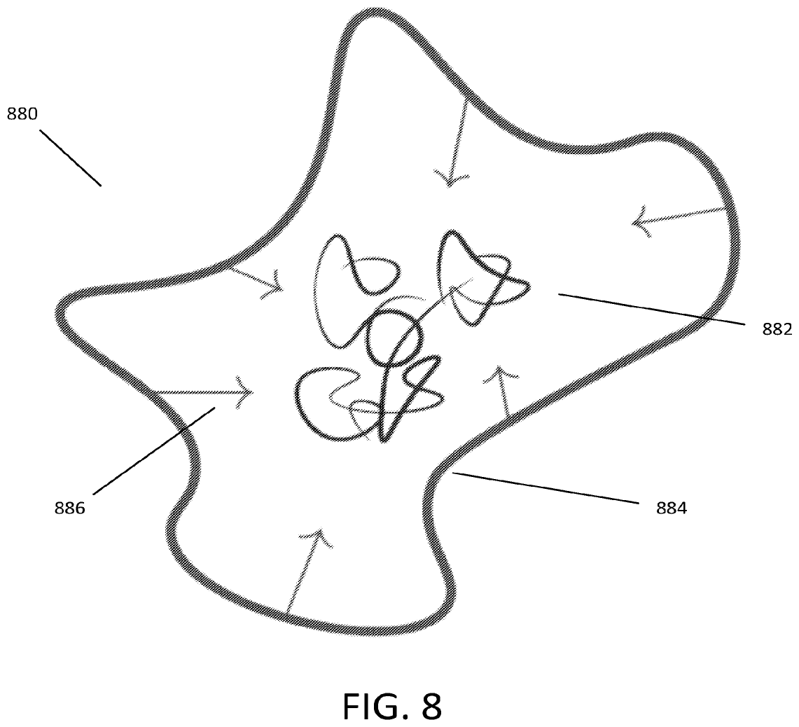

Deep energy estimator networks. arXiv preprint arXiv:1805.08306, 2018.) In some implementations, the problem is further simplified by learning the energy function on the feature space X=R^{dX} of the GNN (here dX=4018). Instead of learning a probabilistic model for the independent and identically distributed (i.i.d.) sequence G₁ . . . G_{N}, the representation of the sequence in the feature space X₁ . . . X_{N} is studied instead (see FIG. 7). This simplification is due to technical reasons, since the denoising methodology of empirical Bayes is formulated in the Euclidean space where specifically the isotropic Gaussian N(0, σ²I_{d}) that defines the noise model plays a central role. Geometrically, the model is designed such that the negative gradient of the energy function evaluated on the noisy data is directed towards the clean data (see FIG. 8). In other words, learning can be viewed as “shaping” the energy function φ such that −∇φ (known as the score function, see, e.g., Aapo Hyvärinen. Estimation of non-normalized statistical models by score matching. Journal of Machine Learning Research, 6 (Apr): 695-709, 2005.) points toward the data manifold.

FIG. 8 is a schematic representation of a DEEN process 880, in which samples from the random variable X(in the feature space of molecules) are represented by squiggly patterns 882 within an outer shape, noisy samples 884 from Y=X+N(0,σ²I_{d}) are represented by the outer shape 884, and the arrows 886 represent −∇φ(y), where φ is the energy function implemented by a neural network. DEEN's learning objective, in a typical implementation, is a denoising objective rooted in empirical Bayes: the idea is to shape the energy function such that the arrows point to the data manifold.

Some technical aspects of an implementation of DEEN are reviewed next. Consider the Gaussian noise model and corrupt the i.i.d. samples X₁ . . . X_{N} as follows:

Yᵢⱼ=Xᵢ+∈ⱼ, where ∈ⱼ˜N(0,σ²I_{d}) (10)

An important result in empirical Bayes is that the Bayes estimator of X, given a noisy measurement Y=y, is given by x′(y)=x+σ²∇ log p(y), where ∇ is the gradient with respect to the noisy data y, and p(y) is the probability density function of the random variable Y=X+N(0,σ²I_{d}). A key step in DEEN is to parameterize the energy function of Y with a neural network φ∂:R^{dX}→R, where the Bayes estimator takes the parametric form:

x_{∂}(y)=y−σ²∇φ_{∂}(y). (11)

Since the Bayes estimator is the least-squares estimator, the learning objective follows immediately:

[symbol]

(∂)= [symbol] _{(x,y)}∥x−\hat{x}_{∂}( [symbol] )∥₂², (12)

where the expectation is over the empirical distribution over the samples (Xᵢ, Yᵢⱼ) (see Eq. 10) and ∥•∥₂² is the Euclidean norm. The main appeal of the algorithm is that it transforms learning the energy function into an optimization problem because it sidesteps posterior inference or MCMC approximations in latent variable models (see, e.g., Martin J Wainwright and Michael I Jordan. Graphical models, exponential families, and variational inference. Foundations and Trends in Machine Learning, 1 (1-2): 1-305, 2008.) At optimality, given a neural network with sufficient capacity, and enough unlabeled samples (n>>1), finding a good approximation to the energy function: φ(y, ∂*)≈−log p(y) is theoretically guaranteed (modulo a constant).

Summarizing, in terms of learning the distribution of graph-valued molecules, in a typical implementation, up to three simplifications are made:
1. The problem is transformed into learning the distribution in the Euclidean space X, defined by the GNN classifier. This simplification is indeed well-suited in this context and is in line with the desire of having the molecules generated by MCTS be classified as inhibitors.
2. The statistical model is unnormalized, i.e., the energy function is defined modulo an additive constant. This is allowed since only the difference between energies appear in the reward function (introduced next), and the constant cancels out.
3. The algorithm is designed around learning the smoothed density associated with noisy data with the hyperparameter a. This relaxation may be key for regularizing the learning (Saremi and Hyvärinen (2019)), but not critical in the MCTS reward function as will become clear.
   Monas-S: Designing Candidate Inhibitors with MCTS

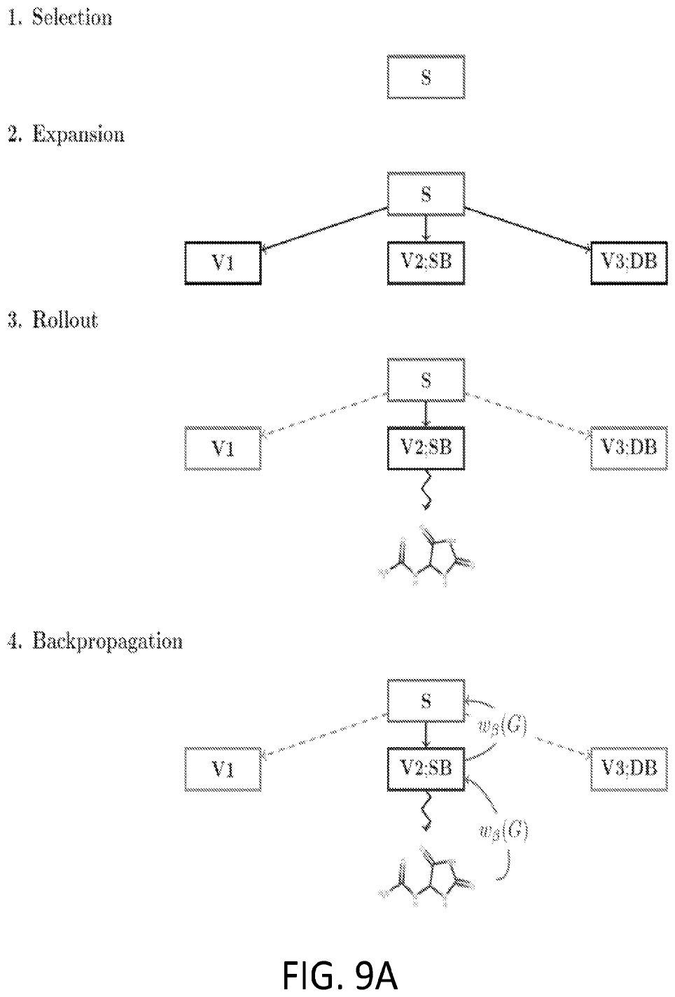

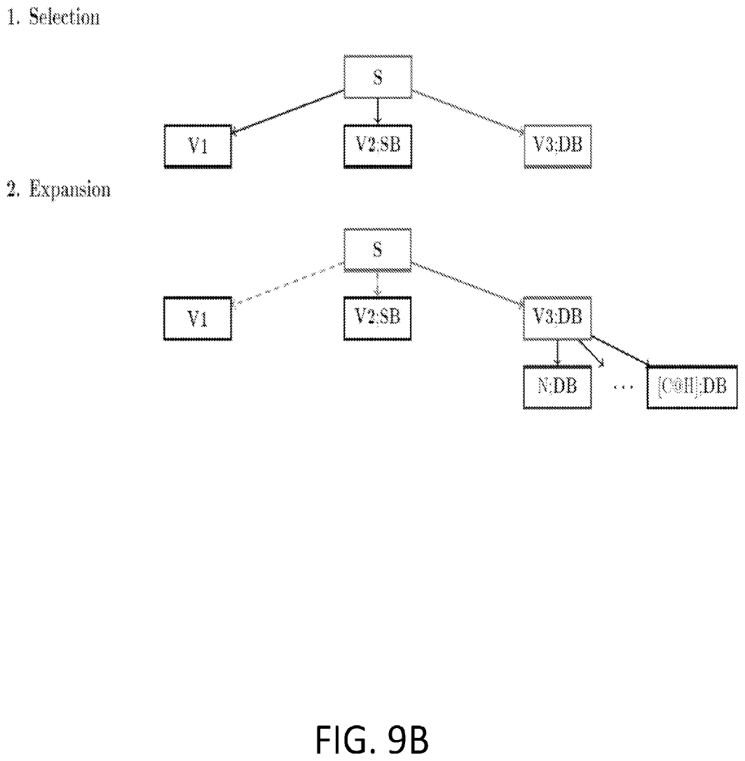

In some implementations, in the automatic design of new inhibitors, a conventional Upper Confidence Trees (UCT) variant of MCTS (see, e.g., Levente Kocsis and Csaba Szepesvári. Bandit based monte Carlo planning. In European conference on machine learning, pages 282-293. Springer, 2006) that searches over a Backus-Naur form (“BNF”) grammar of SMILES strings (see, e.g., Egor Kraev. Grammars and reinforcement learning for molecule optimization. arXiv preprint arXiv:1811.11222, 2018) is used. A detailed description of an exemplary algorithm can be found in Cameron B Browne, Edward Powley, Daniel Whitehouse, Simon M Lucas, Peter I Cowling, Philipp Rohlfshagen, Stephen Tavener, Diego Perez, Spyridon Samothrakis, and Simon Colton. A survey of monte Carlo tree search methods. IEEE Trans. Computational Intelligence and AI in games, 4 (1): 1-43, 2012. MCTS searches a tree of derivations, i.e., sequences of steps which generate valid SMILES strings. In some implementations, in each iteration of MCTS, the following steps are taken (FIG. 9A depicts each of the four steps schematically):
1. Selection: starting from a root node with an associated initial non-terminal (‘S’), successive child nodes are selected. In some implementations, the selections are done according to the UCB1 formula and continue until a previously unvisited node is encountered. In FIG. 9A the root node is initially unexpanded, so it is selected.
2. Expansion: the encountered node is expanded according to the possible productions, e.g., from Leftmost parsing, yielding three children, ‘V1’, ‘V2;SB’ and ‘V3;DB’, in the case of our example. One of the new children (‘V2;SB’) is selected, for example, at random. If the encountered node has no viable children, it represents a completed SMILES string, and is instead simply evaluated.
3. Rollout: a random derivation is generated for the selected child, such that the partially completed SMILES string at the selected node is completed at random yielding a valid SMILES string. In some implementations, the rollout is performed uniformly at random, except when a terminal symbol is available; in which case decisions are made uniformly at random over available terminal symbols. The completed SMILES string is then converted into a molecular representation and evaluated using the reward function w.
4. Backpropagation: the evaluated reward ω_{β} of the molecule G is backpropagated through the tree. In a typical implementation, every node that was visited has its average reward wᵢ and maximum reward w^{max} updated. Additionally, the number of visits to each visited node n is incremented.

FIG. 9B represents an alternative example of a process like the one in FIG. 9A, but that includes only a selection step and an expansion step.

As a reminder of the general method, at least according to an exemplary implementation:
The method generates SMILES strings as derivations in a grammar.
To do this, it treats the space of possible derivations as a tree. Every node in the tree represents a decision point where a decision must be made over which derivation to follow. The possible options are given by productions e.g., rules in the grammar. A production involves replacing a non-terminal with other non-terminals and/or terminals. The SMILES string is complete once it contains no more non-terminals.
The method uses MCTS to search over SMILES strings.
During the steps of MCTS, the computer is doing:
   Selection. The computer points towards the root/start node of the tree of derivations. It follows a path down through the tree according to the modified WCB1 formula until it reaches a node that has been fully expanded.
   Expansion. The computer generally uses the partially completed SMILES string at the unexpanded node alongside the grammar to generate all possible productions. These productions are added as child nodes of the previously unexpanded node, and one of these child nodes is selected at random.
   Rollout. The computer takes the partially completed SMILES string at the randomly chosen child-node and completes it by selected random productions from the options available according to the grammar. This produces a completed SMILES string which is then evaluated by the computed using the reward function which itself uses MONAS-P and MONAS-E.
   Backpropagation. The computer updates the average and maximum reward values of all nodes visited along the path chosen during the selection stage, using the reward value computed from the rollout.

Moreover, to clarify certain terms that relate to this portion of the description:
Node: a node in a tree e.g., a point where a decision must be made over the possible productions from a grammar.
Child node: Every node has a parent node e.g., a node which must be visited to reach it. A child node is just the inverse of this relationship.
Derivation: A sequence of nodes/productions taken to generate a SMILES string in the grammar.
Non-terminal: An abstract symbol which must be replaced via a production in the grammar so to produce valid SMILES strings. Any string which contains non-terminals is not a valid SMILES string.
Possible productions: The possible rules from the grammar which can be applied.
Leftmost parsing: Non-terminals that appear towards the left-hand-side of the smiles string typically are replaced first.
Viable children: Synonym for possible productions, but with respect to the tree of possible derivations.
Average reward: The average of all of the reward values observed from molecules produced by choosing the given node.
Maximum reward: Same as average reward, but taken as the maximum value observed, rather than an average.

Additionally, a reward function is a function to maximize (see earlier discussion on search), e.g., we want to find derivations of molecules SMILES strings which maximize the reward function as these are likely to be inhibitors (MONAS-P) and structurally similar to the dataset (MONAS-E).

In some implementations, the system 100 utilizes a mildly adjusted version of the UCB1 (Upper Confidence Bound 1 applied to trees) formula (see, e.g., Peter Auer, Nicolò Cesa-Bianchi, and Paul Fischer. Finite-time analysis of the multiarmed bandit problem. Machine learning, 47 (2-3): 235-256, 2002) to determine the appropriate node/production to use in each step of the selection process. The modified UCB1 formula used is:

$$\frac{{{w}_{i}}^{max} + \underline{{w}_{i}}}{2} + c \sqrt{\frac{ln {N}_{i}}{{n}_{i}}} \qquad ( 13 )$$

where for node i, wᵢ^{max} is the maximum reward observed, wᵢ is the average reward observed, nᵢ is the total number of samples, Nᵢ is the total number of samples at the parent of node i, and c is the exploration/exploitation parameter. The introduction of a maximum reward term wᵢ^{max} is simply to help the system pursue particularly promising samples which are otherwise avoided due to a few poor-quality samples from that same node. Once all non-terminals have been cleared either by selection or rollout, a complete SMILES string is generated for evaluation. As stated, the rollout policy typically is random, but prioritizes terminals where possible in order to bias our sampling towards smaller molecules.

Typically, every time MCTS generates a SMILES string, it is converted to a molecule and then a graph, as described above. By passing the sample molecule G through the GNN, a prediction of inhibition ƒ_{A}(G) is obtained for assay A, and a latent representation X of the molecule which is further passed through the DEEN model to yield an energy φ(X). With φₘᵢₙ defined as the minimum energy for any known inhibitor in the test set, the reward returned to MCTS is then

$${ω}_{β} ( G ) = {f}_{A} ( G ) \cdot \frac{2}{1 + exp ( βΔφ ( X ) )} , with Δφ ( X ) = φ ( X ) - {φ}_{min} \qquad ( 14 )$$

meaning that the sample molecule is heavily penalized for either a low inhibition prediction or high energy. The β term is a hyperparameter controlling the smoothness of the energy component of the reward function, with a default value of

$${β}_{0} = \frac{1}{{φ}_{max} - {φ}_{min}} \qquad ( 15 )$$

(maximum and minimum typically are computed by the system 100 over the test set). A simple but important property of the reward function is that it is invariant under φ=φ+c. The reward function is generally invariant to this transformation since the energy function itself is defined modulo an additive constant.

### Experiments

In the following discussion, an automatic molecule design framework/process comprises three steps:
1. The ensemble of GNNs is trained to predict inhibition for the four assays.
2. DEEN is then trained on the feature space of molecules generated by the ensemble of GNNs to learn an energy model of the dataset.
3. The GNN ensemble and the DEEN model are used in the reward function (Eq. 14) to guide MCTS, generating potential novel inhibitors.

To further boost performance, given the relatively small number of positive samples, a simple Bootstrap-Aggregating ensemble of models (see, e.g., Leo Breiman. Bagging predictors. Machine learning, 24 (2): 123-140, 1996) was used in which the outputs of the ensemble members are averaged to generate predictions. To build the ensemble, the training data is split into five non-overlapping folds of equal size. Each model is trained on a different set of four folds in a standard cross-fold manner, yielding five models which have been trained under different conditions. For each model, the unused fold serves as a validation set. The parameters of each at the epoch at which the validation loss is lowest is used to construct the final ensemble.

The data was first split into two disjoint subsets: 80% of the data training, 20% for testing. The training data is then split into five folds, and a network is trained on each subset of four folds, yielding five models. The various subsets of data are stratified such that each contains approximately the same number of positive and negative samples for each task. Each model was trained using the ADAM optimizer (see, e.g., Diederik P Kingma and Jimmy Ba. Adam: A method for stochastic optimization. arXivpreprint arXiv:1412.6980, 2014), with an initial learning rate of 2×10⁻⁵, batch size of 128 and weight decay of 1×10⁻⁴. Each model was trained until the validation loss did not improve for 20 epochs. Here, the validation loss is computed on the unused fold for each model.

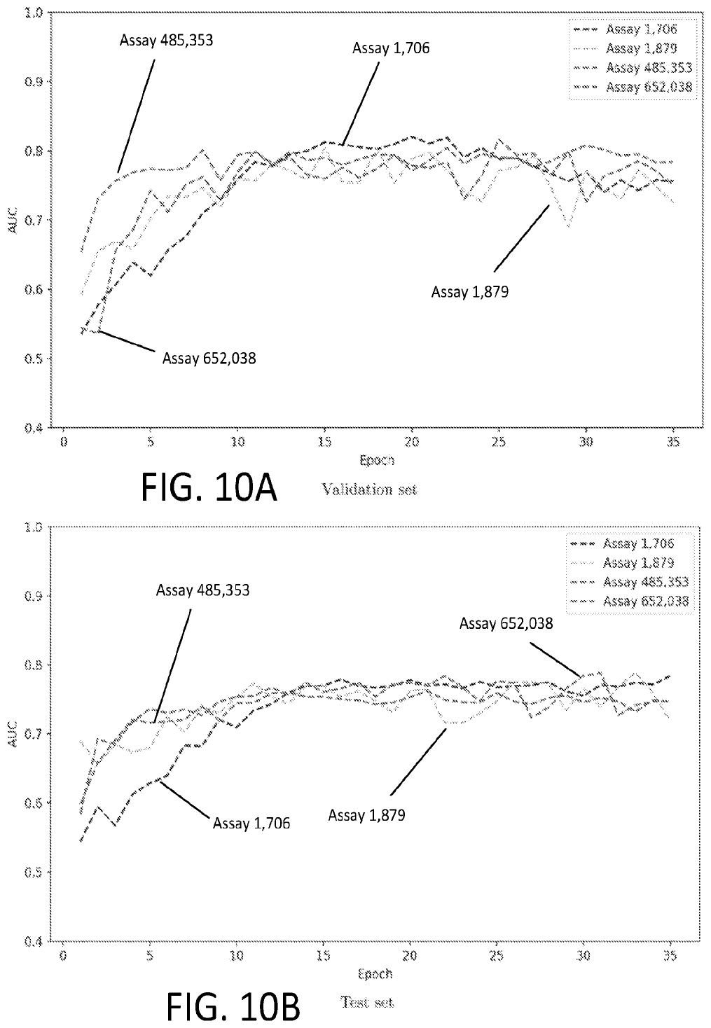

A typical training cycle is represented in FIGS. 10A and 10B, which respectfully show. validation and testing receiver operator characteristic (ROC) area under curve (AUC) scores of model 0 (see Table 2), measured during training and reported for each assay. The lowest validation loss for this model was observed at epoch 15. A full listing of ROC AUC scores of the models is given in Table 2, alongside those of the ensemble computed by averaging the outputs of the member models. The ensemble achieves higher scores than the average score of its individual members across all assays. In general, the ensemble has a 75-80% likelihood of ranking an inhibitor higher than a non-inhibitor across all tasks. Although there is room for further improvement, meaningful classification performance is clearly demonstrated.

TABLE 2

| | Termination | Assay ROC AUC Score | | | |
|---|---|---|---|---|---|
| Model | Epoch | 1,708 | 1,879 | 485,353 | 652,038 |
| 0 | 15 | 0.77 | 0.77 | 0.76 | 0.76 |
| 1 | 15 | 0.77 | 0.83 | 0.74 | 0.78 |
| 2 | 21 | 0.77 | 0.83 | 0.77 | 0.79 |
| 3 | 22 | 0.76 | 0.77 | 0.72 | 0.79 |
| 4 | 16 | 0.75 | 0.78 | 0.76 | 0.79 |
| Average | 17.8 | 0.76 | 0.79 | 0.75 | 0.78 |
| Ensemble | — | 0.78 | 0.81 | 0.76 | 0.80 |

In Table 2, for each assay, the score of the best performing model is highlighted in bold. The average (mean) scores are provided in comparison that of the ensemble as a whole. For every assay, the ensemble performs better on the test data than the average of its individual members. In two assays (1,708 and 652,038), the ensemble performs better than any of the individual GNNs.

To validate the MONAS-P architecture, it was compared empirically with five other ensembles, each using a different type of GNN found in molecular chemistry literature: Message Passing Neural Networks (MPNNs; see, e.g., Justin Gilmer, Samuel S Schoenholz, Patrick F Riley, Oriol Vinyals, and George E Dahl. Neural message passing for quantum chemistry. arXiv preprint arXiv:1704.01212, 2017), Directed Message Passing Neural Networks (D-MPNN; see, e.g., Kevin Yang, Kyle Swanson, Wengong Jin, Connor Coley, Philipp Eiden, Hua Gao, Angel Guzman-Perez, Timothy Hopper, Brian Kelley, Miriam Mathea, et al. Analyzing learned molecular representations for property prediction. Journal of chemical information and modeling, 59 (8): 3370-3388, 2019), Attentive FingerPrints (Attentive FP; see, e.g., Zhaoping Xiong, Dingyan Wang, Xiaohong Liu, Feisheng Zhong, Xiaozhe Wan, Xutong Li, Zhaojun Li, Xiaomin Luo, Kaixian Chen, Hualiang Jiang, et al. Pushing the boundaries of molecular representation for drug discovery with the graph attention mechanism. Journal of Medicinal Chemistry, 63 (16): 8749-8760, 2020), and the full Graph Networks architecture of Battaglia et al. (2018) with global conditioning. Certain implementation details for each method are provided below.

The results of these comparisons are shown in Table 3, all for the same reserved subset of test data.

TABLE 3

| | Assay ROC AUC Score | | | | |
|---|---|---|---|---|---|
| Model | 1,708 | 1,879 | 485,353 | 652,038 | Average |
| MPNN [16] | 0.75 | 0.78 | 0.74 | 0.78 | 0.76 |
| D-MPNN [52] | 0.74 | 0.71 | 0.67 | 0.81 | 0.73 |
| Attentive FP [51] | 0.76 | 0.75 | 0.73 | 0.80 | 0.76 |
| Graph Networks [2] | 0.76 | 0.79 | 0.73 | 0.80 | 0.77 |
| MONAS-P | 0.78 | 0.81 | 0.76 | 0.80 | 0.79 |

Table 3 provides an inhibition prediction performance comparison. For each assay the best performing model's ROC AUC score is highlighted in bold. Each result is reported for a bagging ensemble of five models each trained on different sub-sets of the training data. As can be seen, MONAS-P outperforms all other approaches studied, except on assay 652,038 where it is comparable to Attentive FP and Graph Networks and is marginally outperformed by D-MPNN. Furthermore, MONAS-P provides the highest ROC AUC score averaged across all four assays, confirming that it is well-suited to the problem. Note that the intent of these experiments is not to conduct an exhaustive empirical comparisons on this task but is instead to provide context and verify that MONAS-P is both appropriate and at least comparable to other state-of-the-art techniques.

### Parameters for GNN Variants in Compared in Table 3

All GNN variants were trained using the Adam optimizer and the loss function described herein. Each ensemble of networks is trained using the same five folds of training data, with each member of the ensemble using a different fold as a validation set for early stopping. In each case the final model chosen is that which achieved the lowest loss on its validation set, and early stopping is determined when the validation loss has not decreased for 20 epochs. For each network variant, the hyper-parameters were chosen by hand-tuning the parameter settings that were either described in the respective publications or found in authors' publicly available code.

### MPNN, Gilmer et al. (2017), Parameters

Node dimensionality=200
Edge dimensionality=50
Layers T=3
Number of Set2Set applications=3
Batch size=20
Learning rate=10^{-2.9}

Every 50000 batches, the learning rate is halved.

### D-MPNN, Yang et al. (2019), Parameters

Hidden dimensionality=300
Number of Directed Message Passing layers T=6
Output MLP layers F=6
Batch size=50
Learning rate=10⁻⁴

Every 50000 batches, the learning rate is halved.

### Attentive FP, Xiong et al. (2020), Parameters

Radius=3
T=2
Fingerprint dimensionality=150
Dropoutp=0.1
Batch size=100
Learning rate=10^{-3.5}
Weight decay=10^{-2.9}

### Graph Network, Battaglia et al. (2018), Parameters

Node, edge and global dimensionality=96
Number of message passing layers T=3
Output MLP layers F=2
Dropout on output layers p=0.25 and p=0.5, respectively
Batch size=128,
Learning rate=2×10⁻⁵
Weight decay=10⁻⁴

### Training DEEN

According to an exemplary implementation, DEEN is trained on the feature space of the training molecules with Gaussian noise with a standard deviation (σ) of 0.25. The 574-dimensional latent representations taken across all five members of the ensemble in this example are concatenated by the system 100 to yield a single 2880-dimensional latent representation (labeled Xin the Section about MONAS-E, above). An MLP was used with four layers of 3072,2048 and 1024 neurons, respectively, followed by a single linear output neuron. Each layer, except for the first and last layer, uses a skip connection so that it takes, as input, the concatenation of the two previous layer outputs. All neurons use the smoothed ReLU activation function u/(1+exp(−u)) which is known by two different names: SiLU and Swish. The choice of a smooth activation function is important in this example due to the double backpropagation (one in y to compute the loss, the other in 8 to compute the gradient of the loss) for a single SGD update. The ADAM optimizer is again used, with a learning rate of 10⁻⁵ and a batch-size of 128. DEEN is trained for 200 epochs.

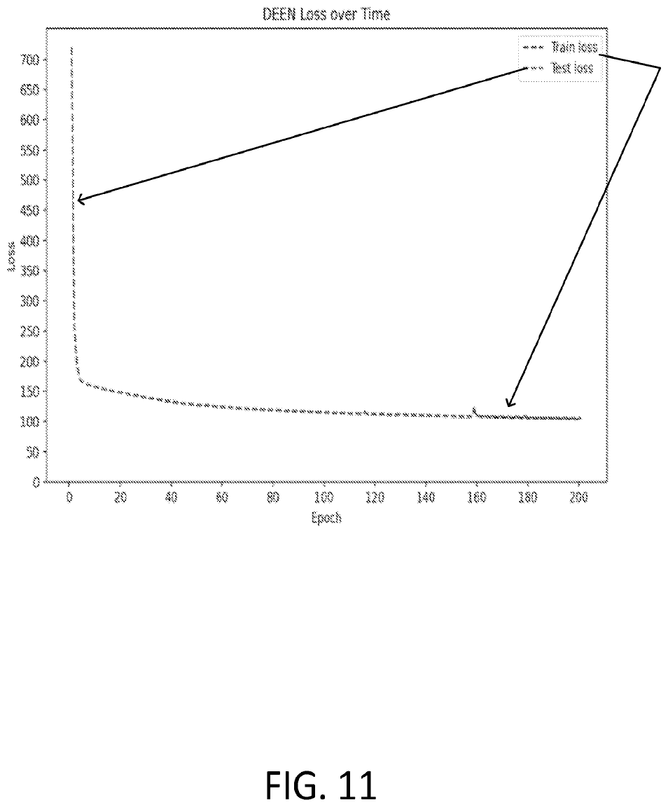

FIG. 11 shows DEEN model learning curves that indicate the training and testing loss measured during the 200 training epochs. The epoch with the lowest testing loss is the final epoch. Note that the test loss closely follows the training loss, suggesting that the testing data lies on the same manifold of latent representations as the training data.

### Discovering Novel Inhibitors

In an exemplary implementation, for each assay, MCTS optimizes the reward function for one million samples. This process is repeated ten times, yielding a total search of ten million molecules per assay. Once MCTS has terminated, in an exemplary implementation, the top 30K unique sampled molecules are identified for each assay, where uniqueness is determined by comparing canonical SMILES strings. Additionally, in order to bias search toward smaller molecules, any derivation branch which reaches a depth ≥30 immediately prioritizes terminals to pursue termination of the derivation. The exploration constant c is set to \sqrt{2}.

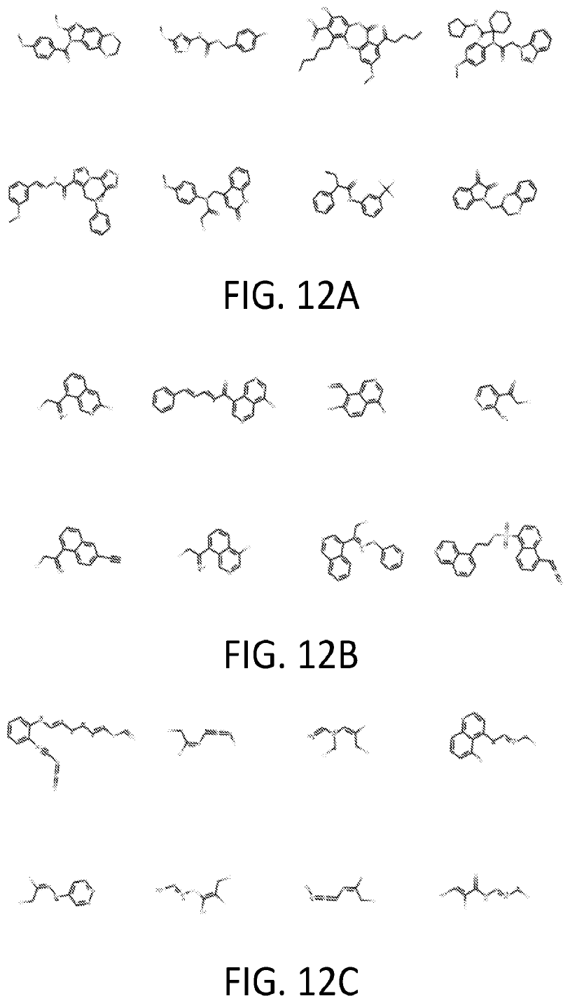

FIGS. 12A-12C shows eight known inhibitors for assay 1,706 in comparison to eight inhibitors discovered with and without energy regularization. More specifically, FIG. 12A shows eight known inhibitors (e.g., from the dataset of SARS-CoV-1 inhibition 332), FIG. 12B shows eight inhibitors discovered with energy regularization (where R does not equal 0), and FIG. 12C shows eight inhibitors discovered without energy regularization (where R equals 0). These result clearly indicates the importance of the energy model in the reward function. The inhibitors discovered with DEEN (with energy regularization) enabled (FIG. 12B) closely resemble molecules found within the dataset (FIG. 12A). In contrast, those inhibitors (FIG. 12C) discovered without energy regularization (0=0) are often unusually simple and linear.

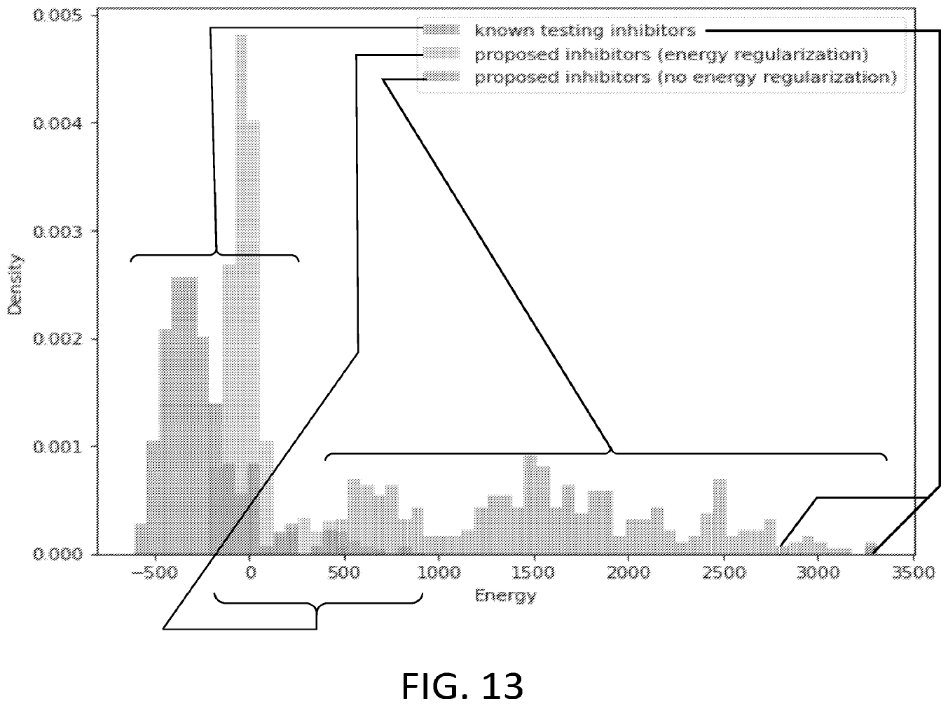

To highlight this point, FIG. 13 compares the energies (as computed by the energy model) of various discovered inhibitors with and without energy regularization and known (testing) inhibitors. Many of the proposed inhibitors generated without energy regularization are far from the learned manifold of the known inhibitors, while the distribution of inhibitors discovered with regularization are centered within the range of known-inhibitor energy values. In fact, they are much more concentrated than the known inhibitors, suggesting that perhaps the best explored inhibitors lie within a similar subspace of the overall combinatorial space of molecules.

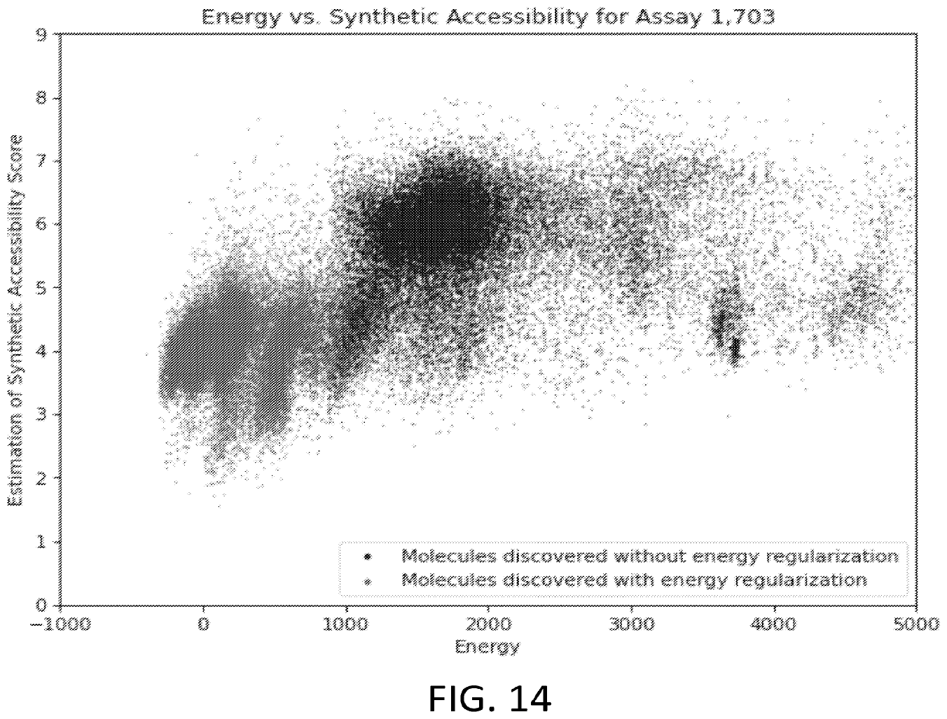

FIG. 14 demonstrates that the energy model has also captured the general “synthesizability” property of the original dataset. In this figure, the estimation of synthetic accessibility score (see, e.g., Peter Ertl and Ansgar Schuffenhauer. Estimation of synthetic accessibility score of drug-like molecules based on molecular complexity and fragment contributions. Journal of cheminformatics, 1 (1): 8, 2009) of each discovered potential inhibitor is plotted against energy for assay 1,706, both with (lighter dots) and without (darker dots) energy regularization. Molecules discovered with regularization tend to have substantially lower estimation of synthetic accessibility scores, meaning that it is more likely that they are practically synthesizable.

A number of embodiments of the invention have been described. Nevertheless, it will be understood that various modifications may be made without departing from the spirit and scope of the invention.

For example, much of this document focuses on designing novel molecules for inhibiting SARS-CoV-1 and SARS-CoV-2. The systems and techniques disclosed herein, however, may be used for a wide variety of data-driven molecular optimization or design tasks.

In various implementations, certain computer components disclosed herein (e.g., the property predictor (MONAS-P), the energy model (MONAS-E), and the search engine (MONAS-S)) may be implemented by one or more computer-based processors (collectively, processor) executing computer-readable instructions stored on non-transitory computer-readable media to perform their respective associated computer-based functionalities. The one or more computer-based processors can be virtually any kind of computer-based processors. They can be contained in one housing or distributed at different locations. Moreover, the non-transitory computer-readable media can be or include any one or more of a variety of different computer-based hardware memory/storage devices either contained in one housing or distributed at different locations.

An effective way to implement the systems and techniques disclosed herein involves the use of Graph Neural Networks (GNNs) as MONAS-P, as these neural network models are permutation invariant. In some implementations, the system may be configured to exploit the hidden state space of the trained GNN from MONAS-P and use this as a bootstrapped vector representation on which to train an energy model. There are a number of viable search procedures to use as MONAS-S, although one viable method is to use Monte-Carlo Tree Search in a grammar to explore derivations of molecules' SMILES string representations. An alternative, which would readily support multi-objective search, is to use a graph-based genetic algorithm, alongside a multi-objective evolutionary algorithm such as the nondominated sorting genetic algorithm, NSGA II.

The systems and techniques may be implemented/performed using one or more computers specially programmed to perform the functionalities disclosed herein. Some of the work performed in connection with this document was carried out on an Ubuntu 20.04 desktop machine with the following hardware specifications: 32 GB DDR4 RAM, an AMD Ryzen 9 3900× 12-core processor, a NVIDIA GeForce RTX 2080 SUPER, and an MSI MPG X570 GamingPro Carbon WiFi Motherboard. Moreover, some of the work was completed in Python 3.7 as the primary programming language, with the libraries Pytorch and Torch Geometric providing interfaces to GPU accelerated functionality.

Certain user functionalities (e.g., viewing results, initiating design processes, entering data, etc.) may be accessible or activated by a user viewing and/or interacting with onscreen elements (e.g., windows, buttons or the like).

It should be understood that the example embodiments described herein may be implemented in many different ways. In some instances, the various methods and machines described herein may each be implemented by a physical, virtual, or hybrid general purpose computer, such as the computer system, or the computer network environment described herein. The computer system may be transformed into the machines that execute the methods described herein, for example, by loading software instructions into either memory or non-volatile storage for execution by the CPU. One of ordinary skill in the art should further understand that the system and its various components may be configured to carry out any embodiments or combination of embodiments of the present invention described herein. Further, the system may implement the various embodiments described herein utilizing any combination of hardware, software, and firmware modules operatively coupled, internally, or externally, to or incorporated into the system.

Various aspects of the subject matter disclosed herein can be implemented in digital electronic circuitry, or in computer-based software, firmware, or hardware, including the structures disclosed in this specification and/or their structural equivalents, and/or in combinations thereof. In some embodiments, the subject matter disclosed herein can be implemented in one or more computer programs, that is, one or more modules of computer program instructions, encoded on computer storage medium for execution by, or to control the operation of, one or more data processing apparatuses (e.g., processors). Alternatively, or additionally, the program instructions can be encoded on an artificially generated propagated signal, for example, a machine-generated electrical, optical, or electromagnetic signal that is generated to encode information for transmission to suitable receiver apparatus for execution by a data processing apparatus. A computer storage medium can be, or can be included within, a computer-readable storage device, a computer-readable storage substrate, a random or serial access memory array or device, or a combination thereof. While a computer storage medium should not be considered to be solely a propagated signal, a computer storage medium may be a source or destination of computer program instructions encoded in an artificially generated propagated signal. The computer storage medium can also be, or be included in, one or more separate physical components or media, for example, multiple CDs, computer disks, and/or other storage devices.

Certain operations described in this specification (e.g., aspects of those represented in FIGS. 5 and 6, and otherwise disclosed herein) can be implemented as operations performed by a data processing apparatus (e.g., a processor/specially-programmed processor) on data stored on one or more computer-readable storage devices or received from other sources, such as the computer system and/or network environment in FIGS. 1, 2, and/or 3. The term “processor” (or the like) encompasses all kinds of apparatuses, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, for example, code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. Moreover, the term “processor” should be construed broadly to include any one or more computer-based processors.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be described herein as occurring in a particular order or manner, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Other implementations are within the scope of the claims.

What is claimed is:

1. A computer system for designing molecules with a desired property, the computer system comprising:
a computer-based processor; and
a computer-readable medium storing computer-readable instructions that, when executed by the computer-based processor, cause the computer-based processor to embody:
a computer-based search engine configured to search for proposed molecules;
a computer-based property predictor to predict a likelihood of the desired property being present in the proposed molecules;
a computer-based energy model to approximate a degree of similarity between the proposed molecules and molecules known to possess the desired property,
wherein the search is guided by the property predictor's predictions and the energy model's approximations.

2. The computer system of claim 1, further comprising:
a dataset of training molecules in the computer-readable medium,
wherein the dataset of training molecules contains data regarding the molecules known to possess the desired property.

3. The computer system of claim 2, wherein the computer-based property predictor has been trained, in a supervised manner, to predict the likelihood of the desirable property being present in the proposed molecules, using the dataset of training molecules.

4. The computer system of claim 3, wherein the computer-based energy model has been trained, in an unsupervised manner, to model a distribution of data in the dataset of training molecules.

5. The computer-system of claim 4, wherein, for each molecule that the computer-based search engine proposes:
the computer-based property predictor predicts a likelihood that the proposed molecule has the desired property; and
the computer-based energy model approximates a degree to which the proposed molecule is similar to molecules represented in the dataset of training molecules.

6. The computer system of claim 5, further comprising:
a dataset of proposed molecules in the computer-readable medium,
wherein, for each molecule proposed by the search engine, the system determines whether to store data associated with that proposed molecule in the dataset of proposed molecules, based on the predicted likelihood that that proposed molecule has the desired property, and based on the approximate degree to which that proposed molecule is similar to the molecules represented in the dataset of training molecules.

7. The computer system of claim 6, wherein the search engine scores each respective one of the proposed molecules based on the predicted likelihood of that proposed molecule having the desired property, and based on the approximate degree to which that proposed molecules is similar to the molecules represented in the dataset of training molecules; and
storing the data associated with that proposed molecule in the dataset of proposed molecules if the score for that proposed molecule is above a threshold score or higher than assigned composite scores for other proposed molecules.

8. The computer system of claim 6, wherein the computer-based property predictor is a computer-based graph neural network (GNN), the energy model is a Deep Energy Estimator Network (“DEEN”), and the search engine executes Monte Carlo tree searching (MCTS).

9. The computer system of claim 1, wherein the desired property is inhibition of SARS-CoV-1.

10. A computer-based method for designing molecules having a desired property, the computer-based method comprising:
providing a computer system comprising:
a computer-based processor; and
a computer-readable medium storing computer-readable instructions that, when executed by the computer-based processor, cause the computer-based processor to embody:
a computer-based property predictor to predict a likelihood that each respective one of a plurality of proposed molecules has the desired property;
a computer-based energy model to approximate a statistical similarity between each respective one of the proposed molecules and a set of molecules represented in a stored dataset of training molecules, wherein the dataset of training molecules contains data regarding a set of molecules known to possess the desired property; and
a computer-based search engine to conduct a search through a space of candidate molecules to propose molecules likely to have the desired property, wherein the search is guided by the property predictor's predictions and by the energy model's approximations.

11. The computer-based method of claim 10, further comprising:
storing the dataset of training molecules in the computer-readable medium; and
training, in a supervised manner, the computer-based property predictor to predict a likelihood that the desirable property is present in a molecule using the dataset of training molecules.

12. The computer-based method of claim 11, further comprising:
training, in an unsupervised manner, the energy model to model a distribution of data regarding the training molecules in the dataset of training molecules.

13. The computer-based method of claim 12, further comprising:
conducting the search through the space of candidate molecules with the computer-based search engine guided by the property predictor's predictions and the energy model's approximations.

14. The computer-based method of claim 13, further comprising, for each molecule that the computer-based search engine proposes:
predicting, with the computer-based property predictor, a likelihood that that proposed molecule has the desired property; and
approximating, with the computer-based energy model, a degree to which that proposed molecule is similar to molecules represented in the dataset of training molecules.

15. The computer-based method of claim 14, further comprising:
storing, in the computer-readable medium, a dataset of proposed molecules; and
for each molecule that the computer-based search engine proposes:
determining whether to store data associated with that proposed molecule in the dataset of proposed molecules, based on the predicted likelihood that that proposed molecule has the desired property and the approximate degree to which that proposed molecule is similar to the molecules represented in the dataset of training molecules.

16. The computer-based method of claim 15, further comprising:
scoring each respective one of the proposed molecules based on the predicted likelihood of that proposed molecule having the desired property and the approximate degree to which that proposed molecules is similar to the molecules represented in the dataset of training molecules; and
storing the data associated with that proposed molecule in the dataset of proposed molecules based on the score for that proposed molecule.

17. The computer-based method of claim 16, wherein the computer-based property predictor is a computer-based graph neural network (GNN), the energy model is a Deep Energy Estimator Network (“DEEN”), and the search engine executes Monte Carlo tree searching (MCTS).

18. The computer-based method of claim 17, wherein the desired property is inhibition of SARS-CoV-1.

19. A non-transitory computer readable medium having stored thereon computer-readable instructions that, when executed by a computer-based processor, cause the computer-based processor to embody:
a computer-based property predictor that predicts a likelihood that each respective one of a plurality of proposed molecules has the desired property;
a computer-based energy model that approximates a statistical similarity between each respective one of the proposed molecules and a set of molecules represented in a stored dataset of training molecules, wherein the dataset of training molecules contains data regarding a set of molecules known to possess the desired property; and
a computer-based search engine that conducts a search through a space of candidate molecules to propose molecules likely to have the desired property, wherein the search is guided by the property predictor's predictions and by the energy model's approximations.
